(19) United States
(12) Patent Application Publication
(10) Pub. No.: US 2025/0292400 A1
(43) Pub. Date: Sep. 18, 2025

(54) IMAGE PROCESSING DEVICE, ENDOSCOPE SYSTEM, IMAGE PROCESSING METHOD, AND PROGRAM

(71) Applicant: FUJIFILM CORPORATION, Tokyo (JP)

(72) Inventor: Toshihiro USUDA, Kanagawa (JP)

(21) Appl. No.: 19/047,628

(22) Filed: Feb. 7, 2025

(30) Foreign Application Priority Data
Mar. 12, 2024 (JP) 2024-038214

(51) Int. Cl. G06T 7/00; A61B 8/00; A61B 8/06; G06V 10/60

(52) U.S. Cl. CPC G06T 7/0012; A61B 8/06; A61B 8/488; G06V 10/60; G06T 2207/10132; G06T 2207/30096

(57) ABSTRACT

The image processing device includes a processor. The processor acquires a first recognition result obtained by executing recognition processing of inputting an ultrasound image, which is obtained by a B-mode method in an ultrasound diagnosis for a subject and in which an observation target region of the subject is shown, to a trained model to cause the trained model to recognize the observation target region. The processor outputs a second recognition result in which the first recognition result is changed based on blood flow distribution information obtained by a Doppler method used in combination with the B-mode method in the ultrasound diagnosis.

### CROSS-REFERENCE TO RELATED APPLICATIONS

This application claims priority under 35 USC 119 from Japanese Patent Application No. 2024-038214, filed on Mar. 12, 2024, the disclosure of which is incorporated by reference herein.

### BACKGROUND

### 1. Technical Field

The present disclosure relates to an image processing device, an endoscope system, an image processing method, and a program.

### 2. Related Art

JP2021-159696A discloses an ultrasound diagnostic device comprising an acquisition unit and a display control unit. In the ultrasound diagnostic device described in JP2021-159696A, the acquisition unit transmits ultrasound to a scan region in a subject, and acquires a B-mode image in which a signal intensity of a reflected wave reflected in the subject is represented by a brightness of luminance, and a Doppler image of a region of interest included in the scan region. In the ultrasound diagnostic device described in JP2021-159696A, the display control unit displays the Doppler image on a display unit in a manner of being superimposed on the B-mode image acquired by the acquisition unit. In the ultrasound diagnostic device described in JP2021-159696A, the display control unit changes a display state of the Doppler image to be displayed on the display unit using a feature amount obtained based on at least one of time-series B-mode images or Doppler images acquired by the acquisition unit.

JP2016-047474A discloses an ultrasound diagnostic device comprising a transmission control unit, a detection unit, an evaluation unit, and a display image control unit. In the ultrasound diagnostic device described in JP2016-047474A, the transmission control unit transmits first ultrasound to a biological tissue from an ultrasound probe, and transmits second ultrasound to the ultrasound probe after the first ultrasound is transmitted. In the ultrasound diagnostic device described in JP2016-047474A, the detection unit detects the presence or absence of movement of an object in the biological tissue by the first ultrasound in a two-dimensional region based on an echo signal obtained by the transmission of the second ultrasound. In the ultrasound diagnostic device described in JP2016-047474A, the evaluation unit evaluates tissue properties for a portion of interest of the biological tissue based on the detection by the detection unit. In the ultrasound diagnostic device described in JP2016-047474A, the display image control unit displays an image according to the evaluation by the evaluation unit. The processing of detecting the presence or absence of movement of the object by the detection unit is color Doppler processing or B-flow processing, and the evaluation unit performs evaluation according to a size of a region in which Doppler data is obtained by the color Doppler processing or a region in which B-flow data is obtained by the B-flow processing.

WO2020/110500A discloses an ultrasound diagnostic device comprising a gate setting unit, a Doppler processing unit, a display unit, and an image enlargement unit. In the ultrasound diagnostic device described in WO2020/110500A, the gate setting unit sets a Doppler gate in a blood vessel region by performing image analysis on a B-mode image in which at least the blood vessel region is imaged. In the ultrasound diagnostic device described in WO2020/110500A, the Doppler processing unit generates a Doppler waveform image based on Doppler data in the Doppler gate. In the ultrasound diagnostic device described in WO2020/110500A, the display unit displays the B-mode image and the Doppler waveform image. In the ultrasound diagnostic device described in WO2020/110500A, in a case where both the B-mode image and the Doppler waveform image are frozen by a user, the image enlargement unit displays an enlarged B-mode image obtained by enlarging the blood vessel region including the Doppler gate on the display unit.

### SUMMARY

One embodiment according to the present disclosure provides an image processing device, an endoscope system, an image processing method, and a program capable of, in a case where erroneous recognition of an observation target region occurs due to recognition processing using a trained model for an ultrasound image obtained by a B-mode method, outputting a correct recognition result and suppressing outputting of an erroneous recognition result.

A first aspect according to the present disclosure relates to an image processing device comprising: a processor, in which the processor acquires a first recognition result obtained by executing recognition processing of inputting an ultrasound image, which is obtained by a B-mode method in an ultrasound diagnosis for a subject and in which an observation target region of the subject is shown, to a trained model to cause the trained model to recognize the observation target region, and outputs a second recognition result in which the first recognition result is changed based on blood flow distribution information obtained by a Doppler method used in combination with the B-mode method in the ultrasound diagnosis.

A second aspect according to the present disclosure relates to the image processing device according to the first aspect, in which the observation target region is a lesion, and the second recognition result is information obtained by changing the first recognition result such that the lesion and a portion other than the lesion are distinguishable from each other.

A third aspect according to the present disclosure relates to the image processing device according to the second aspect, in which the lesion is a cyst.

A fourth aspect according to the present disclosure relates to the image processing device according to the second or third aspect, in which the blood flow distribution information is a map capable of specifying an intensity of a blood flow, and the second recognition result is information in which the first recognition result is changed based on a portion in the map where the intensity is equal to or greater than a reference intensity.

A fifth aspect according to the present disclosure relates to the image processing device according to any one of the second to fourth aspects, in which the trained model is obtained by performing machine learning for recognizing the lesion.

A sixth aspect according to the present disclosure relates to the image processing device according to any one of the second to fifth aspects, in which the second recognition result is information capable of distinguishing the lesion from the portion other than the lesion.

A seventh aspect according to the present disclosure relates to the image processing device according to any one of the second to sixth aspects, in which the outputting of the second recognition result includes displaying the second recognition result on a first screen as visible information capable of distinguishing the lesion from the portion other than the lesion.

An eighth aspect according to the present disclosure relates to the image processing device according to the first aspect, in which the observation target region is a blood vessel, the blood vessel is specified from the blood flow distribution information, and the second recognition result is information obtained by changing the first recognition result such that the blood vessel and a portion other than the blood vessel are distinguishable from each other.

A ninth aspect according to the present disclosure relates to the image processing device according to the eighth aspect, in which a lesion other than the blood vessel is included, and the lesion is a cyst.

A tenth aspect according to the present disclosure relates to the image processing device according to the eighth or ninth aspect, in which the blood flow distribution information is a map capable of specifying an intensity of a blood flow, and the second recognition result is information in which the first recognition result is changed based on a portion in the map where the intensity is less than a reference intensity.

An eleventh aspect according to the present disclosure relates to the image processing device according to any one of the eighth to tenth aspects, in which the trained model is obtained by performing machine learning for recognizing the blood vessel.

A twelfth aspect according to the present disclosure relates to the image processing device according to any one of the eighth to eleventh aspects, in which the second recognition result is information capable of distinguishing the blood vessel from the portion other than the blood vessel.

A thirteenth aspect according to the present disclosure relates to the image processing device according to any one of the eighth to twelfth aspects, in which the outputting of the second recognition result includes displaying the second recognition result on a first screen as visible information capable of distinguishing the blood vessel from the portion other than the blood vessel.

A fourteenth aspect according to the present disclosure relates to the image processing device according to any one of the first to thirteenth aspects, in which the ultrasound image is displayed on a second screen in a foreground, and the recognition processing is executed and the first recognition result is changed to the second recognition result in a background.

A fifteenth aspect according to the present disclosure relates to the image processing device according to any one of the first to fourteenth aspects, in which, in a case where processing of changing the first recognition result to the second recognition result based on the blood flow distribution information is being executed, the processor outputs information capable of specifying that the processing of changing the first recognition result to the second recognition result based on the blood flow distribution information is being executed.

A sixteenth aspect according to the present disclosure relates to an endoscope system comprising: the image processing device according to any one of the first to fifteenth aspects; and an ultrasound probe that emits ultrasound and detects a reflected wave of the ultrasound in a state in which the ultrasound probe is inserted into a body of the subject, in which the ultrasound image and the blood flow distribution information are generated based on the reflected wave detected by the ultrasound probe.

A seventeenth aspect according to the present disclosure relates to an image processing method comprising: acquiring a first recognition result obtained by executing recognition processing of inputting an ultrasound image, which is obtained by a B-mode method in an ultrasound diagnosis for a subject and in which an observation target region of the subject is shown, to a trained model to cause the trained model to recognize the observation target region, and outputting a second recognition result in which the first recognition result is changed based on blood flow distribution information obtained by a Doppler method used in combination with the B-mode method in the ultrasound diagnosis.

An eighteenth aspect according to the present disclosure relates to a program for causing a computer to execute a process comprising: acquiring a first recognition result obtained by executing recognition processing of inputting an ultrasound image, which is obtained by a B-mode method in an ultrasound diagnosis for a subject and in which an observation target region of the subject is shown, to a trained model to cause the trained model to recognize the observation target region, and outputting a second recognition result in which the first recognition result is changed based on blood flow distribution information obtained by a Doppler method used in combination with the B-mode method in the ultrasound diagnosis.

### BRIEF DESCRIPTION OF THE DRAWINGS

Exemplary embodiments of the technology of the disclosure will be described in detail based on the following figures, wherein:

FIG. 1 is a conceptual diagram showing an example of an aspect in which an endoscope system is used;

FIG. 2 is a conceptual diagram showing an example of an overall configuration of the endoscope system;

FIG. 3 is a block diagram showing an example of a configuration of an ultrasound endoscope;

FIG. 4 is a conceptual diagram showing an example of an aspect in which a cyst recognition model is generated by causing a model to learn a B-mode image group;

FIG. 5 is a conceptual diagram showing an example of a processing content of a generation unit;

FIG. 6 is a conceptual diagram showing an example of a processing content by a processor in a case where erroneous recognition of a cyst occurs (here, as an example, in a case where a blood vessel is erroneously recognized as a cyst);

FIG. 7 is a flowchart showing an example of a flow of diagnosis support processing.

FIG. 8 is a flowchart showing an example of a flow of background processing;

FIG. 9 is a flowchart showing an example of a flow of foreground processing;

FIG. 10 is a conceptual diagram showing an example of an aspect in which a blood vessel recognition model is generated by causing a model to learn a B-mode image group;

FIG. 11 is a conceptual diagram showing an example of a processing content by the processor in a case where the blood vessel is erroneously recognized (here, as an example, in a case where the cyst is erroneously recognized as the blood vessel);

FIG. 12 is a flowchart showing a modification example of the flow of the diagnosis support processing;

FIG. 13 is a conceptual diagram showing an example of an aspect in which information indicating that erroneous recognition suppression processing is being executed is displayed on a screen; and

FIG. 14 is a conceptual diagram showing an example of a series of pieces of processing in which a processor included in a computer gives a processing execution request to an external device via a network, the external device executes processing according to the processing execution request, and the processor included in the computer receives a processing result from the external device.

### DETAILED DESCRIPTION

Hereinafter, an example of embodiments of an image processing device, an endoscope system, an image processing method, and a program according to the present disclosure will be described with reference to the accompanying drawings.

First, terms used in the following description will be described.

CPU is an abbreviation for “central processing unit”. GPU is an abbreviation for “graphics processing unit”. TPU is an abbreviation for “tensor processing unit”. RAM is an abbreviation for “random-access memory”. EEPREEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electro-luminescence”. CMOS is an abbreviation for “complementary metal-oxide-semiconductor”. CCD is an abbreviation for “charge-coupled device”. LAN is an abbreviation for “local area network”. WAN is an abbreviation for “wide area network”. AI is an abbreviation for “artificial intelligence”. BLI is an abbreviation for “blue light imaging”. LCI is an abbreviation for “linked color imaging”. NN is an abbreviation for “neural network”. CNN is an abbreviation for “convolutional neural network”. R-CNN is an abbreviation for “region based convolutional neural network”. YOLO is an abbreviation for “you only look once”. RNN is an abbreviation for “recurrent neural network”. FCN is an abbreviation for “fully convolutional network”. FIFO is an abbreviation for “first in first out”.

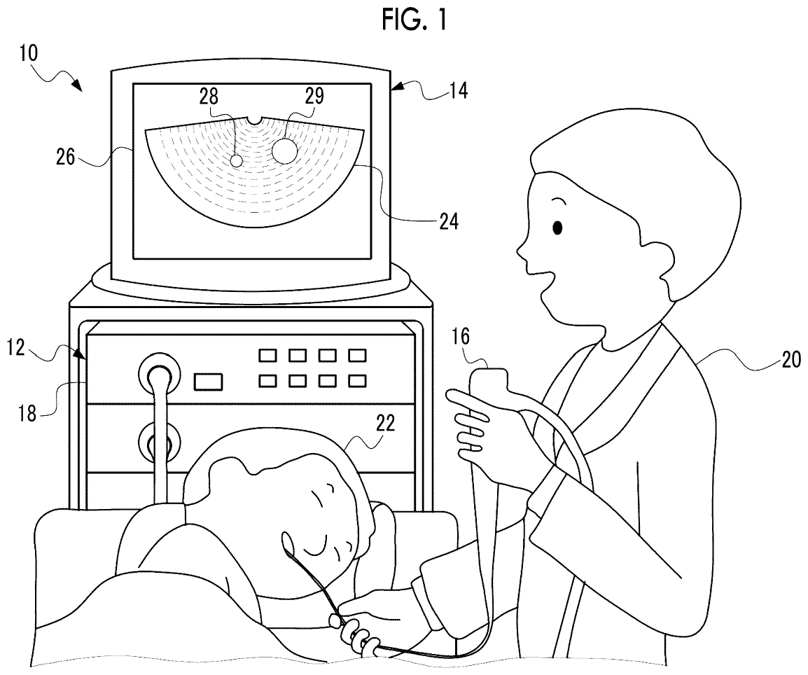

As shown in FIG. 1 as an example, an endoscope system 10 comprises an ultrasound endoscope 12 and a display device 14. The ultrasound endoscope 12 is a convex type ultrasound endoscope, and comprises an ultrasound endoscope body 16 and a processing device 18. In the present embodiment, the endoscope system 10 is an example of an “endoscope system” according to the present disclosure. In addition, in the present embodiment, the processing device 18 is an example of an “image processing device” according to the present disclosure.

It should be noted that, in the present embodiment, although the convex type ultrasound endoscope is shown as an example of the ultrasound endoscope 12, this is merely an example, and the present disclosure is established even in a case of a radial type ultrasound endoscope. In addition, in the present embodiment, although the ultrasound endoscope 12 is shown as an example, this is merely an example, and the present disclosure is established even in a case of an extracorporeal ultrasound diagnostic device.

The endoscope system 10 is used by a user 20. An example of the user 20 is a doctor. The ultrasound endoscope body 16 is connected to the processing device 18, and transmits and receives various signals to and from the processing device 18. That is, the processing device 18 outputs a signal to the ultrasound endoscope body 16 to control an operation of the ultrasound endoscope body 16, or performs various types of signal processing on a signal input from the ultrasound endoscope body 16.

The ultrasound endoscope 12 is an endoscope used for ultrasound diagnosis on a subject 22 (for example, a patient). In the ultrasound diagnosis on the subject 22, in a case where the user 20 observes an internal part (for example, a pancreas) that is a part present inside a body of the subject 22, the user 20 inserts the ultrasound endoscope body 16 into the body of the subject 22 from a mouth or a nose of the subject 22 (in the example shown in FIG. 1, the mouth). Then, in a state in which the ultrasound endoscope body 16 is inserted into the body of the subject 22, the ultrasound endoscope body 16 emits ultrasound to the internal part at a predetermined position (for example, a position of a stomach or a duodenum) of the body of the subject 22, and detects a reflected wave obtained by the emitted ultrasound being reflected at the internal part. The processing device 18 generates an ultrasound image 24 based on the reflected wave detected by the ultrasound endoscope body 16, and outputs the ultrasound image 24 to the display device 14 or the like.

It should be noted that the example shown in FIG. 1 shows an aspect in which an upper gastrointestinal endoscopy is performed, but the present disclosure is not limited to this, and the present disclosure can also be applied to a lower gastrointestinal endoscopy, a bronchoscopy, and the like.

The display device 14 displays various types of information including an image under the control of the processing device 18. Examples of the display device 14 include a liquid crystal display and an EL display. The ultrasound image 24 generated by the processing device 18 is displayed as a moving image on a screen 26 of the display device 14. In the example shown in FIG. 1, a cyst 28 and a blood vessel 29 are shown in the ultrasound image 24 displayed on the screen 26. In the present embodiment, the screen 26 is an example of a “first screen” and a “second screen” according to the present disclosure.

It should be noted that the example shown in FIG. 1 shows a form example in which the ultrasound image 24 is displayed on the screen 26 of the display device 14, but this is merely an example, and the ultrasound image 24 may be displayed on a display device (for example, a display of a tablet terminal) other than the display device 14. The ultrasound image 24 may be stored in a computer-readable non-transitory storage medium (for example, a flash memory, an HDD, and/or a magnetic tape).

The display device 14 displays an image according to a type of an ultrasound diagnostic method, which is a diagnostic method using ultrasound. A type of an ultrasonography method is selected by the user 20 or is selected according to various conditions. Examples of the type of the ultrasonography method include a brightness mode (B-mode) method and a Doppler method (for example, a color Doppler method).

The B-mode method is an ultrasound diagnosis method that generates and displays a B-mode image. The B-mode image refers to a two-dimensional tomographic image obtained in a cross section parallel to a direction of ultrasound propagation and is obtained by converting an intensity of the reflected wave, obtained by the ultrasound emitted to the internal part being reflected at the internal part, into a brightness on the screen 26.

The Doppler method is an ultrasound diagnosis method that generates and displays a Doppler image. The Doppler image refers to an image that shows blood dynamics in a blood vessel with color information by using the reflected wave, obtained by the ultrasound emitted to the internal part being reflected at the internal part. The image showing the blood dynamics in the blood vessel is, for example, an image expressed by red-based and blue-based colors. The red-based color is a color indicating a blood flow toward a side where the ultrasound is emitted, and the blue-based color is a color indicating a blood flow away from the side where the ultrasound is emitted. In a case where a reflected wave having a higher frequency than the ultrasound that hits the blood flow is obtained, the blood flow is specified as a blood flow directed toward the side where the ultrasound is emitted, whereas in a case where a reflected wave having a lower frequency than the ultrasound that hits the blood flow is obtained, the blood flow is specified as a blood flow directed away from the side where the ultrasound is emitted. In the blood vessel, there are a portion where the blood flow is fast and a portion where the blood flow is slow, and in the Doppler image, the speed of the blood flow is expressed as a gradation through color mapping.

On the screen 26, the B-mode image or the Doppler image is displayed as the ultrasound image 24, or the B-mode image and the Doppler image are displayed in parallel, according to the ultrasound diagnostic method selected by the user 20. In the example shown in FIG. 1, the B-mode image is displayed on the screen 26 as the ultrasound image 24. The ultrasound image 24 is a moving image including a plurality of frames generated according to a predetermined frame rate (for example, 30 frames/second). A frame rate of the Doppler method is lower than a frame rate of the B-mode method. An example of the frame rate of the Doppler method (that is, the frame rate at which the Doppler image is generated) is 15 frames/second. Here, although the moving image is shown as an example, this is merely an example, and the present disclosure is established even in a case where the ultrasound image 24 is a still image. In the following description, in order to facilitate understanding of the present disclosure, the description will be made on the premise that the ultrasound image 24 is the B-mode image.

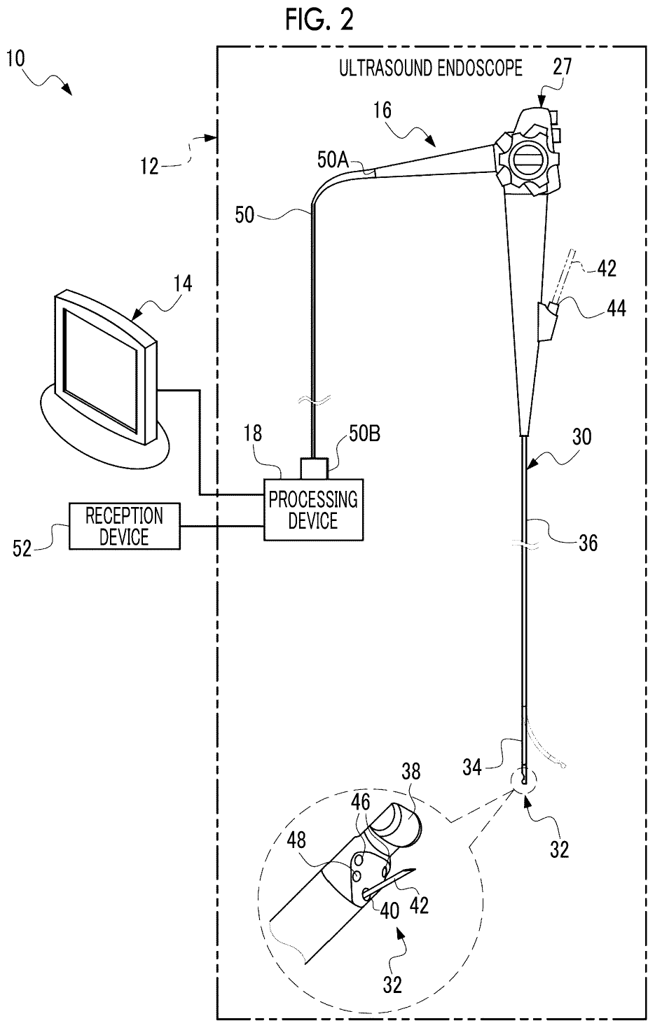

As shown in FIG. 2 as an example, the ultrasound endoscope body 16 comprises an operating part 27 and an insertion part 30. The insertion part 30 is formed in a tubular shape. The insertion part 30 includes a distal end part 32, a bendable part 34, and a flexible part 36. The distal end part 32, the bendable part 34, and the flexible part 36 are disposed in an order of the distal end part 32, the bendable part 34, and the flexible part 36 from a distal end side to a base end side of the insertion part 30. The flexible part 36 is formed of an elongated flexible material and connects the operating part 27 and the bendable part 34. The bendable part 34 is partially bent or rotated about an axial center of the insertion part 30 by operating the operating part 27. As a result, the insertion part 30 is sent to a back side of a luminal organ while being bent in accordance with a shape of the luminal organ (for example, a shape of a duodenal tract) or being rotated about an axis of the insertion part 30.

The distal end part 32 is provided with an ultrasound probe 38 and a treatment tool opening 40. The ultrasound probe 38 is provided on a distal end side of the distal end part 32. The ultrasound probe 38 is a convex type ultrasound probe, and in a state of being inserted into the body of the subject 22, emits the ultrasound to the internal part and receives the reflected wave, obtained by the emitted ultrasound being reflected at the internal part.

The treatment tool opening 40 is formed on a base end side of the distal end part 32 with respect to the ultrasound probe 38. The treatment tool opening 40 is an opening for allowing a treatment tool 42 to protrude from the distal end part 32. A treatment tool insertion port 44 is formed at the operating part 27, and the treatment tool 42 is inserted into the insertion part 30 through the treatment tool insertion port 44. The treatment tool 42 passes through the insertion part 30 and protrudes from the treatment tool opening 40 to an outside of the ultrasound endoscope body 16. The treatment tool opening 40 also functions as a suction port for suctioning blood, internal waste, and the like.

In the example shown in FIG. 2, a puncture needle is shown as the treatment tool 42. It should be noted that this is merely an example, and the treatment tool 42 may be forceps and/or a cannula.

An illumination device 46 and a camera 48 are provided in the distal end part 32. The illumination device 46 applies light. Examples of a type of light applied from the illumination device 46 include visible light (for example, white light), invisible light (for example, near-infrared light), and/or special light. Examples of the special light include light for BLI and/or light for LCI.

The camera 48 images an inside of the luminal organ by an optical method. A CMOS image sensor is used in the camera 48. The CMOS image sensor is merely an example, and other types of image sensors such as a CCD image sensor may be used. The image obtained by being captured by the camera 48 is displayed on the display device 14, is displayed on a display device (for example, a display of a tablet terminal) other than the display device 14, is stored in a storage medium (for example, a flash memory, an HDD, and/or a magnetic tape), or is stored in an electronic medical record.

The ultrasound endoscope 12 comprises the processing device 18 and a universal cord 50. The universal cord 50 has a base end part 50A and a distal end part 50B. The base end part 50A is connected to the operating part 27. The distal end part 50B is connected to the processing device 18. That is, the ultrasound endoscope body 16 and the processing device 18 are connected to each other via the universal cord 50.

The endoscope system 10 comprises a reception device 52. The reception device 52 is connected to the processing device 18. The reception device 52 receives an instruction from a user. Examples of the reception device 52 include an operation panel having a plurality of hard keys and/or a touch panel, a keyboard, a mouse, a trackball, a foot switch, a smart device, and/or a microphone.

The processing device 18 performs various types of signal processing or transmits and receives various signals to and from the ultrasound endoscope body 16 and the like in response to the instruction received by the reception device 52. For example, the processing device 18 emits the ultrasound to the ultrasound probe 38 in response to the instruction received by the reception device 52, generates the ultrasound image 24 (see FIG. 1) based on the reflected wave received by the ultrasound probe 38, and outputs the generated ultrasound image 24.

The display device 14 is also connected to the processing device 18. The processing device 18 controls the display device 14 in response to the instruction received by the reception device 52. As a result, for example, the ultrasound image 24 generated by the processing device 18 is displayed on the screen 26 of the display device 14 (see FIG. 1).

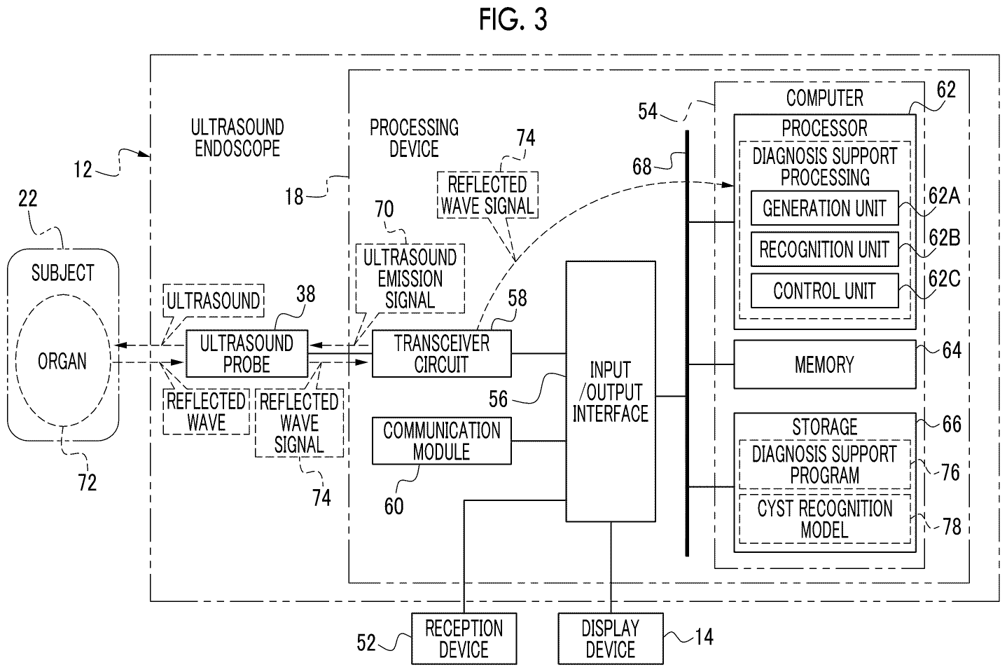

As shown in FIG. 3 as an example, the processing device 18 comprises a computer 54, an input/output interface 56, a transceiver circuit 58, and a communication module 60. The computer 54 is an example of a “computer” according to the present disclosure.

The computer 54 comprises a processor 62, a memory 64, and a storage 66. The input/output interface 56, the processor 62, the memory 64, and the storage 66 are connected to a bus 68.

The processor 62 controls the entire processing device 18. For example, the processor 62 includes a CPU and a GPU, and the GPU is operated under the control of the CPU, and is mainly responsible for executing image processing. It should be noted that the processor 62 may be one or more CPUs with an integrated GPU function or may be one or more CPUs without an integrated GPU function. The processor 62 may include a multi-core CPU or a TPU. In the present embodiment, the processor 62 is an example of a “processor” according to the present disclosure.

The memory 64 is a memory that temporarily stores information, and is used as a work memory by the processor 62. Examples of the memory 64 include a RAM. The storage 66 is a nonvolatile storage device that stores various programs, various parameters, and the like. Examples of the storage 66 include a flash memory (for example, an EEPROM) and/or an SSD. It should be noted that the flash memory and the SSD are merely examples, and the storage 66 may be other nonvolatile storage devices such as an HDD, or may be a combination of two or more types of nonvolatile storage devices.

The reception device 52 is connected to the input/output interface 56, and the processor 62 acquires the instruction received by the reception device 52 via the input/output interface 56 and executes processing in response to the acquired instruction.

The transceiver circuit 58 is connected to the input/output interface 56. The transceiver circuit 58 generates an ultrasound emission signal 70 having a pulse waveform to output the ultrasound emission signal 70 to the ultrasound probe 38 in response to an instruction from the processor 62. The ultrasound probe 38 converts the ultrasound emission signal 70 input from the transceiver circuit 58 into the ultrasound and emits the ultrasound to an organ 72 (for example, a pancreas) as an internal part of the subject 22. The ultrasound probe 38 receives a reflected wave that is obtained by the ultrasound emitted to the organ 72 being reflected at the organ 72, converts the reflected wave into a reflected wave signal 74, which is an electrical signal, and outputs the reflected wave signal 74 to the transceiver circuit 58. The transceiver circuit 58 digitizes the reflected wave signal 74 input from the ultrasound probe 38 and outputs the digitized reflected wave signal 74 to the processor 62 via the input/output interface 56. The processor 62 generates the ultrasound image 24 (see FIG. 1) showing an aspect of the organ 72 based on the reflected wave signal 74 input from the transceiver circuit 58 via the input/output interface 56. In addition, the processor 62 also generates a Doppler image based on the reflected wave signal 74 in parallel with the generation of the ultrasound image 24.

Although not shown in FIG. 3, the illumination device 46 (see FIG. 2) is also connected to the input/output interface 56. The processor 62 controls the illumination device 46 via the input/output interface 56 to change the type of the light applied from the illumination device 46 or to adjust an amount of the light. In addition, although not shown in FIG. 3, the camera 48 (see FIG. 2) is also connected to the input/output interface 56. The processor 62 controls the camera 48 via the input/output interface 56 or acquires the image obtained by imaging the inside of the body of the subject 22 using the camera 48 via the input/output interface 56.

The communication module 60 is connected to the input/output interface 56. The communication module 60 is an interface including a communication processor, an antenna, and the like. The communication module 60 is connected to a network (not shown) such as a LAN or a WAN, and controls communication with an external device (for example, a server, a personal computer, and/or a tablet terminal).

The display device 14 is connected to the input/output interface 56, and the processor 62 controls the display device 14 via the input/output interface 56 such that various types of information are displayed on the display device 14.

The reception device 52 is connected to the input/output interface 56, and the processor 62 acquires the instruction received by the reception device 52 via the input/output interface 56 and executes processing in response to the acquired instruction.

The storage 66 stores a diagnosis support program 76 and a cyst recognition model 78. The processor 62 performs diagnosis support processing by reading out the diagnosis support program 76 from the storage 66 and executing the readout diagnosis support program 76 on the memory 64. The diagnosis support processing is processing of executing recognition processing of recognizing an observation target region from the organ 72 using an AI method, and supporting a diagnosis performed by the user 20 (see FIG. 1) based on a recognition result of the recognition processing. The diagnosis support processing is implemented by the processor 62 operating as a generation unit 62A, a recognition unit 62B, and a control unit 62C in accordance with the diagnosis support program 76 executed on the memory 64.

In the present embodiment, the diagnosis support program 76 is an example of a “program” according to the present disclosure. In addition, the cyst recognition model 78 is a trained model used in the AI method processing of recognizing the cyst 28 shown in the ultrasound image 24. In addition, in the present embodiment, the cyst recognition model 78 is an example of a “trained model” according to the present disclosure.

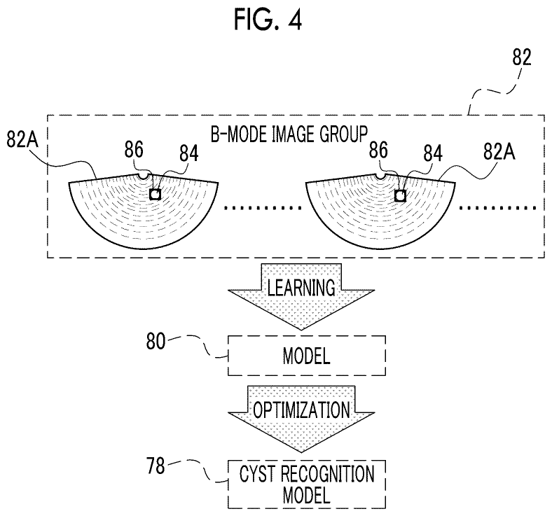

As shown in FIG. 4 as an example, the cyst recognition model 78 is a trained model generated by causing a model 80 to perform machine learning. The cyst recognition model 78 is obtained by performing machine learning for recognizing the cyst 28 (see FIG. 1) shown in the ultrasound image 24 (see FIG. 1). In the machine learning performed on the model 80, a B-mode image group 82 is used as training data. The B-mode image group 82 consists of a plurality of B-mode images 82A different from each other. Examples of the B-mode image 82A include a sample B-mode image obtained by an actual ultrasound diagnosis and/or a sample B-mode image generated by so-called generative AI.

Examples of the model 80 include a mathematical model using an NN. Examples of a type of the NN include a YOLO, an R-CNN, and an FCN. In addition, the NN used in the model 80 may be, for example, the YOLO, the R-CNN, or a combination of the FCN and an RNN. The RNN is suitable for machine learning of a plurality of images obtained in time series. It should be noted that the type of the NN described here is merely an example, and other types of the NNs capable of detecting an object by machine learning of an image may also be used.

The cyst 84 is shown in the plurality of B-mode images 82A. An annotation 86 is assigned to the B-mode image 82A. The annotation 86 is information capable of specifying a position where the cyst 84 is shown in the B-mode image 82A (for example, information including a plurality of coordinates capable of specifying a position of a rectangular frame circumscribing the cyst 84).

Here, for convenience of description, as an example of the annotation 86, information capable of specifying the position where the cyst 84 is shown in the B-mode image 82A is shown, but this is merely an example. For example, the annotation 86 may include various types of information related to a lesion shown in the B-mode image 82A, such as information capable of specifying a type of the lesion shown in the B-mode image 82A, an importance of the lesion, and/or a size of the lesion. Examples of the lesion include a lesion (for example, a tumor) of a type different from the cyst.

It should be noted that, hereinafter, for convenience of description, processing using the cyst recognition model 78 will be described as processing that is actively performed by the cyst recognition model 78 as a main subject. That is, for convenience of description, the cyst recognition model 78 will be described as having a function of performing processing on input information and outputting a processing result. In addition, hereinafter, for convenience of description, a part of processing of training the model 80 will also be described as processing that is actively performed by the model 80 as a main subject. That is, for convenience of description, the model 80 will be described as having a function of performing processing on input information and outputting a processing result.

Each B-mode image 82A included in the B-mode image group 82 is input to the model 80. In response to this, the model 80 recognizes a position where the cyst 84 is shown from the input B-mode image 82A (in other words, predicts the position where the cyst 84 is shown). Then, the model 80 outputs a recognition result (in other words, a prediction result). The recognition result includes information capable of specifying the position recognized by the model 80 as the position where the cyst 84 is shown in the B-mode image 82A. Examples of the information capable of specifying the position recognized by the model 80 include information including a plurality of coordinates capable of specifying a position of a bounding box surrounding a region predicted as a position at which the cyst 84 is present (that is, the position of the bounding box in the B-mode image 82A).

The model 80 is adjusted according to an error between the annotation 86 assigned to the B-mode image 82A input to the model 80 and the recognition result output from the model 80. That is, the model 80 is optimized by adjusting a plurality of optimization variables (for example, a plurality of connection weights and a plurality of offset values) in the model 80 such that the error is minimized, whereby the cyst recognition model 78 is generated. That is, a data structure of the cyst recognition model 78 is obtained by causing the model 80 to learn the plurality of different B-mode images 82A to which the annotations 86 are assigned.

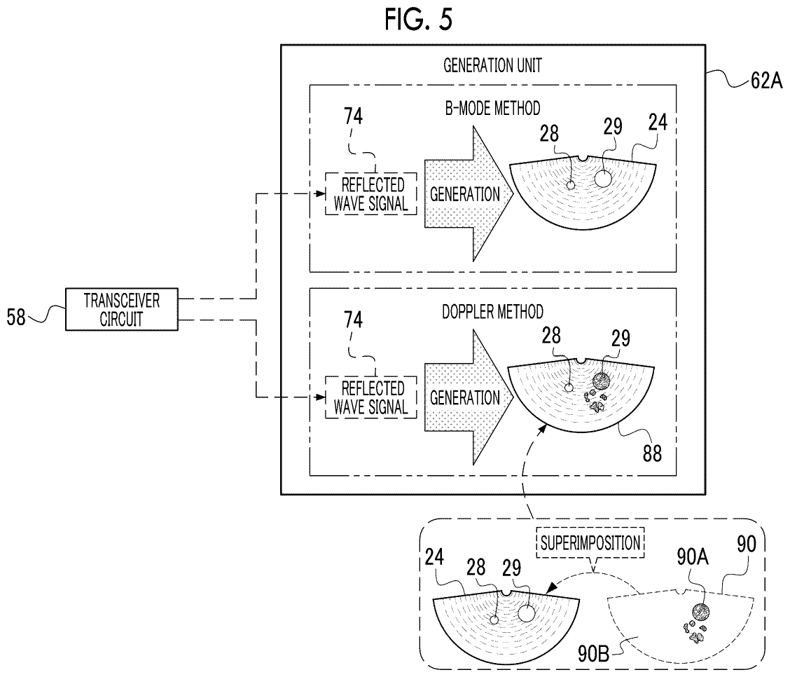

As shown in FIG. 5 as an example, the generation unit 62A acquires the reflected wave signal 74 from the transceiver circuit 58 and generates the ultrasound image 24 using the B-mode method based on the acquired reflected wave signal 74. That is, the generation unit 62A generates a B-mode image based on the reflected wave signal 74 as the ultrasound image 24. The B-mode image generated as the ultrasound image 24 by the generation unit 62A is an image showing a tomogram of the organ 72 (see FIG. 3) in a two-dimensional manner. The example shown in FIG. 5 shows an example in which the ultrasound image 24 in which the cyst 28 and the blood vessel 29 are shown is generated by the generation unit 62A, but as a matter of course, a B-mode image in which the cyst 28 and/or the blood vessel 29 are not shown may be generated as the ultrasound image 24.

In the present embodiment, the ultrasound image 24 generated by the generation unit 62A is an example of an “ultrasound image” according to the present disclosure. In addition, in the present embodiment, the cyst 28 shown in the ultrasound image 24 is an example of an “observation target region of a subject”, a “lesion”, and a “cyst” according to the present disclosure. In addition, in the present embodiment, the blood vessel 29 shown in the ultrasound image 24 is an example of a “blood vessel” according to the present disclosure.

In the present embodiment, in the ultrasound diagnosis on the subject 22, the B-mode method and the Doppler method are used in combination. That is, the generation unit 62A generates a Doppler image 88 in parallel with the ultrasound image 24. Specifically, the generation unit 62A acquires the reflected wave signal 74 used for generating the ultrasound image 24 from the transceiver circuit 58, and generates the Doppler image 88 by the Doppler method based on the acquired reflected wave signal 74. A frame rate of the ultrasound image 24 is higher than a frame rate of the Doppler image 88, and the ultrasound image 24 and the Doppler image 88 are associated with each other in units of a predetermined number of frames of the ultrasound image 24 (for example, in units of 15 frames of the ultrasound image 24). An example of the frame rate of the ultrasound image 24 is 30 frames/second. In addition, an example of the frame rate of the Doppler image 88 is 15 frames/second. It should be noted that this is merely an example, and the frame rate of the ultrasound image 24 may be the same as the frame rate of the Doppler image 88.

The Doppler image 88 is an image obtained by superimposing a color map 90 in which a characteristic (here, as an example, a distribution of blood flow) in the organ 72 is expressed in colors (that is, chromatic colors) on the ultrasound image 24. The color map 90 is information indicating blood flow dynamics specified by using a Doppler effect. In the color map 90, an intensity of the blood flow is expressed in colors. In the present embodiment, a concept of the intensity of the blood flow also includes strength, speed, and/or momentum of the blood flow. The color map 90 includes a blood-flow region 90A indicating a portion where a blood flow is present and a non-blood-flow region 90B indicating a portion where no blood flow is present. From the blood-flow region 90A, the intensity of the blood flow can be specified based on the color applied. Therefore, the image obtained by superimposing the color map 90 on the ultrasound image 24, that is, the Doppler image 88 can also be referred to as a map capable of specifying the intensity of the blood flow. In the present embodiment, the Doppler image 88 is an example of “blood flow distribution information” and a “map” according to the present disclosure.

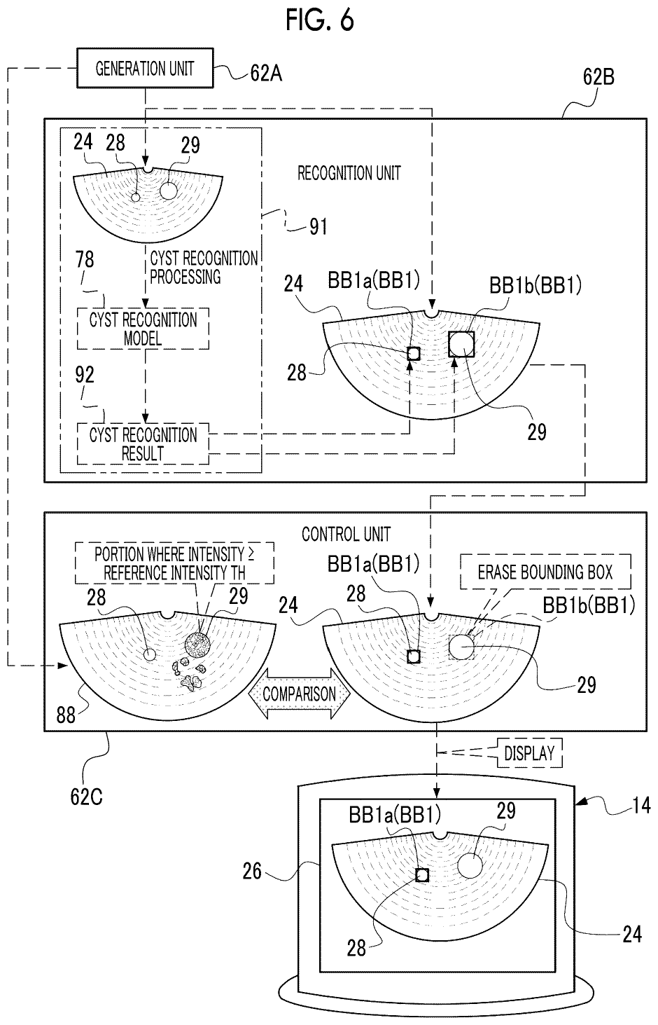

As shown in FIG. 6 as an example, the recognition unit 62B acquires the ultrasound image 24 generated by the generation unit 62A from the generation unit 62A, and executes cyst recognition processing 91 on the acquired ultrasound image 24. The cyst recognition processing 91 is processing of inputting the ultrasound image 24 acquired from the generation unit 62A to the cyst recognition model 78 to cause the cyst recognition model 78 to recognize the cyst 28. In the present embodiment, the cyst recognition processing 91 is an example of “recognition processing” according to the present disclosure.

A cyst recognition result 92 is generated by the cyst recognition model 78 by executing the cyst recognition processing 91. The cyst recognition result 92 includes information capable of specifying the position of the cyst 28 shown in the ultrasound image 24. Here, the cyst recognition result 92 may include a result of erroneously recognizing the blood vessel 29 as the cyst 28.

The recognition unit 62B acquires the cyst recognition result 92 generated by the cyst recognition model 78. The recognition unit 62B reflects the cyst recognition result 92 on the ultrasound image 24 used in the cyst recognition processing 91. In this case, for example, the cyst recognition result 92 is reflected in the ultrasound image 24 as a bounding box BB1. A type of the bounding box BB1 is classified into a first bounding box BB1a and a second bounding box BB1b. The first bounding box BB1a is superimposed on a portion in the ultrasound image 24 where the cyst 28 is shown, by the recognition unit 62B. In the example shown in FIG. 6, the first bounding box BB1a is superimposed on the ultrasound image 24 as a circumscribing frame for the cyst 28 shown in the ultrasound image 24. The second bounding box BB1b is superimposed on a portion in the ultrasound image 24 where the blood vessel 29 is shown, by the recognition unit 62B. The second bounding box BB1b is superimposed on the ultrasound image 24 as a circumscribing frame for the blood vessel 29 shown in the ultrasound image 24.

In addition, in the example shown in FIG. 6, a result of erroneously recognizing the blood vessel 29 as the cyst 28 is superimposed on the ultrasound image 24 as the second bounding box BB1b. Here, in a case where the ultrasound image 24 in which the first bounding box BB1a and the second bounding box BB1b are superimposed is displayed on the screen 26, there is a concern that the user 20 may erroneously recognize the blood vessel 29 as the cyst 28.

Therefore, in the present embodiment, the control unit 62C erases the second bounding box BB1b on the ultrasound image 24. In order to implement this, first, the control unit 62C acquires the ultrasound image 24 in which the first bounding box BB1a and the second bounding box BB1b are superimposed, from the recognition unit 62B. In addition, the control unit 62C acquires the Doppler image 88 (that is, the Doppler image 88 associated with the ultrasound image 24) generated in parallel with the ultrasound image 24 used in the cyst recognition processing 91 from the generation unit 62A, and compares the acquired Doppler image 88 to the ultrasound image 24 in which the first bounding box BB1a and the second bounding box BB1b are superimposed. Then, the control unit 62C specifies a portion (hereinafter, referred to as a “high-intensity portion”) where the intensity of the blood flow is equal to or greater than a reference intensity TH in the Doppler image 88. The reference intensity TH may be a fixed value or a variable value that is changed in accordance with the instruction received by the reception device 52 or the like and/or various conditions. An example of the reference intensity TH in a case where the reference intensity TH is a fixed value includes a predetermined intensity that is generally known as a lower limit of the intensity of the blood flow of the blood vessel 29 present in the organ 72 (here, as an example, the pancreas).

The control unit 62C specifies a portion (hereinafter, referred to as a “high-intensity corresponding portion”) corresponding to the high-intensity portion in the ultrasound image 24 in which the first bounding box BB1a and the second bounding box BB1b are superimposed. In a case where the second bounding box BB1b is superimposed at a position surrounding the specified high-intensity corresponding portion, the control unit 62C erases the second bounding box BB1b. In other words, in the Doppler image 88, in a case where the intensity of the blood flow at the portion corresponding to the portion where the second bounding box BB1b is superimposed is equal to or greater than the reference intensity TH, the control unit 62C erases the second bounding box BB1b. As a result, the ultrasound image 24 is in a state in which only the first bounding box BB1a of the first bounding box BB1a and the second bounding box BB1b is superimposed. This means that the cyst recognition result 92 reflected in the ultrasound image 24 is changed based on the Doppler image 88. That is, it can be said that the first bounding box BB1a remaining on the ultrasound image 24 by erasing the second bounding box BB1b from the ultrasound image 24 is information in which the cyst recognition result 92 is changed based on the high-intensity portion.

The fact that the second bounding box BB1b is erased from the ultrasound image 24 and the first bounding box BB1a remains means that the cyst 28 and portions other than the cyst 28 are distinguishable in the ultrasound image 24. In addition, the fact that the second bounding box BB1b is erased from the ultrasound image 24 and the first bounding box BB1a remains means that the cyst 28 is shown in a portion surrounded by the first bounding box BB1a in the ultrasound image 24, and there is a possibility that the blood vessel 29 is shown in a portion other than the portion surrounded by the first bounding box BB1a in the ultrasound image 24. This means that the cyst recognition result 92 is changed to distinguish between the cyst 28 and the blood vessel 29 based on the Doppler image 88. In other words, it can be said that the first bounding box BB1a remaining on the ultrasound image 24 by erasing the second bounding box BB1b from the ultrasound image 24 is information obtained by changing the cyst recognition result 92 to distinguish between the cyst 28 and the blood vessel 29 based on the Doppler image 88.

The control unit 62C outputs the ultrasound image 24 obtained by erasing the second bounding box BB1b from the ultrasound image 24 in which the first bounding box BB1a and the second bounding box BB1b are superimposed. In the example shown in FIG. 6, the ultrasound image 24 obtained by erasing the second bounding box BB1b from the ultrasound image 24 in which the first bounding box BB1a and the second bounding box BB1b are superimposed is displayed on the screen 26 by the control unit 62C. That is, the first bounding box BB1a superimposed on the ultrasound image 24 is displayed on the screen 26 as visible information capable of distinguishing between the cyst 28 and portions other than the cyst 28.

Here, as an example of outputting the ultrasound image 24 on which the first bounding box BB1a is superimposed, an example in which the ultrasound image 24 on which the first bounding box BB1a is superimposed is displayed on the screen 26 has been described, but the present disclosure is not limited to this. For example, the ultrasound image 24 on which the first bounding box BB1a is superimposed may be stored in a storage region (the storage 66, a server, a personal computer, a tablet terminal, an electronic medical record, or the like), or may be recorded on a medium (for example, paper) by a printer (not shown). In addition, a position of the first bounding box BB1a (that is, a position of the cyst 28) in the ultrasound image 24 may be guided by a voice through a speaker (not shown).

In the present embodiment, the first bounding box BB1a and the second bounding box BB1b superimposed on the ultrasound image 24 are examples of a “first recognition result” according to the present disclosure. In addition, in the present embodiment, the first bounding box BB1a superimposed on the ultrasound image 24 displayed on the screen 26 is an example of a “second recognition result” and “visible information” according to the present disclosure.

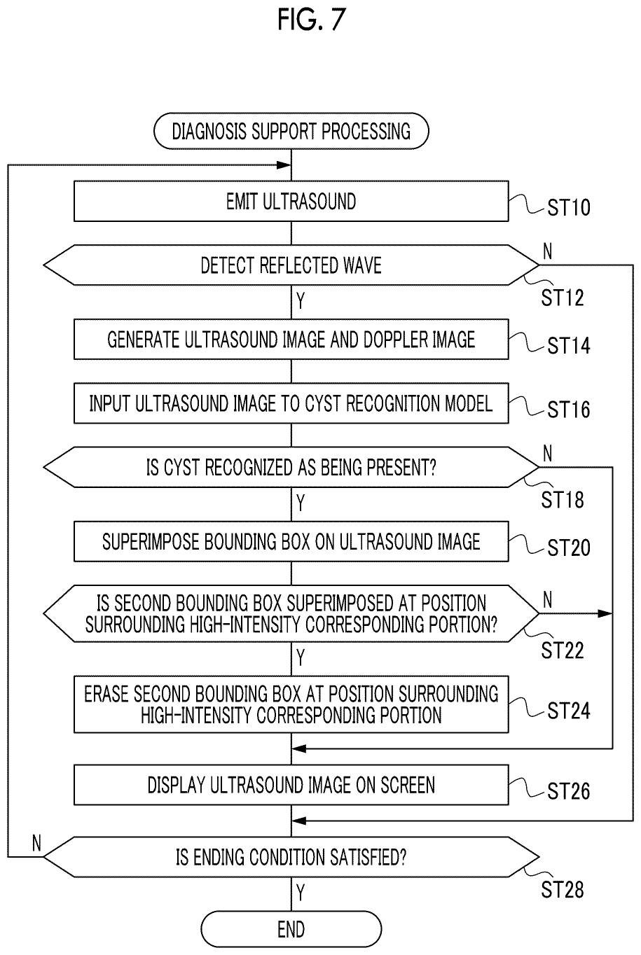

Next, an operation of the endoscope system 10 will be described with reference to FIG. 7.

FIG. 7 shows an example of a flow of the diagnosis support processing performed by the processor 62 of the processing device 18. The flow of the diagnosis support processing shown in FIG. 7 is an example of an “image processing method” according to the present disclosure.

In the diagnosis support processing shown in FIG. 7, first, in step ST10, the generation unit 62A controls the ultrasound probe 38 via the transceiver circuit 58 to cause the ultrasound probe 38 to emit ultrasound to the organ 72 (here, as an example, the pancreas). After the processing of step ST10 is executed, the diagnosis support processing proceeds to step ST12.

In step ST12, the generation unit 62A determines whether or not the reflected wave based on the ultrasound emitted by executing the processing in step ST10 is detected by the ultrasound probe 38. In step ST12, in a case where the reflected wave based on the ultrasound emitted by executing the processing of step ST10 is not detected by the ultrasound probe 38, a negative determination is made, and the diagnosis support processing proceeds to step ST28. In step ST12, in a case where the reflected wave based on the ultrasound emitted by executing the processing of step ST10 is detected by the ultrasound probe 38, a positive determination is made, and the diagnosis support processing proceeds to step ST14.

In step ST14, the generation unit 62A generates the ultrasound image 24 and the Doppler image 88 based on the reflected wave detected by the ultrasound probe 38. After the processing of step ST14 is executed, the diagnosis support processing proceeds to step ST16.

In step ST16, the recognition unit 62B inputs the ultrasound image 24 generated in step ST14 to the cyst recognition model 78. After the processing of step ST16 is executed, the diagnosis support processing proceeds to step ST18.

In step ST18, the recognition unit 62B determines whether or not the cyst 28 is recognized as being shown in the ultrasound image 24 by the cyst recognition model 78 based on the input of the ultrasound image 24 to the cyst recognition model 78. In step ST18, in a case where the cyst 28 is not recognized as being shown in the ultrasound image 24, a negative determination is made, and the diagnosis support processing proceeds to step ST26. In step ST18, in a case where the cyst 28 is recognized as being shown in the ultrasound image 24, a positive determination is made, and the diagnosis support processing proceeds to step ST20.

In step ST20, the recognition unit 62B superimposes the cyst recognition result 92 as the bounding box BB1 on the ultrasound image 24 generated in step ST14. After the processing of step ST20 is executed, the diagnosis support processing proceeds to step ST22.

In step ST22, the control unit 62C determines whether or not the second bounding box BB1b is superimposed at a position surrounding the high-intensity corresponding portion in the ultrasound image 24 by comparing the Doppler image 88 generated in step ST14 to the ultrasound image 24 on which the bounding box BB1 is superimposed. In step ST22, in a case where the second bounding box BB1b is not superimposed at the position surrounding the high-intensity corresponding portion in the ultrasound image 24, a negative determination is made, and the diagnosis support processing proceeds to step ST26. In step ST22, in a case where the second bounding box BB1b is superimposed at the position surrounding the high-intensity corresponding portion in the ultrasound image 24, a positive determination is made, and the diagnosis support processing proceeds to step ST24.

In step ST24, the control unit 62C erases the second bounding box BB1b from the ultrasound image 24 on which the bounding box BB1 is superimposed. After the processing of step ST24 is executed, the diagnosis support processing proceeds to step ST26.

In step ST26, the control unit 62C displays the ultrasound image 24 on the screen 26. That is, in a case where a negative determination is made in step ST18, the ultrasound image 24 generated in step ST14 is displayed on the screen 26 by executing the processing of step ST26. In addition, in a case where a negative determination is made in step ST22, the ultrasound image 24 on which the first bounding box BB1a is superimposed is displayed on the screen 26 by executing the processing of step ST26. Furthermore, in a case where the diagnosis support processing has proceeded to step ST26 after the processing of step ST24 is executed, the ultrasound image 24 on which the first bounding box BB1a is superimposed is displayed on the screen 26 by executing the processing of step ST26. After the processing of step ST24 is executed, the diagnosis support processing proceeds to step ST28.

In step ST28, the control unit 62C determines whether or not a condition for ending the diagnosis support processing is satisfied. Examples of the condition for ending the diagnosis support processing include a condition in which an instruction to end the diagnosis support processing is received by the reception device 52. In step ST28, in a case where the condition for ending the diagnosis support processing is not satisfied, a negative determination is made, and the diagnosis support processing proceeds to step ST10. In step ST28, in a case where the condition for ending the diagnosis support processing is satisfied, the diagnosis support processing is ended.

As described above, in the endoscope system 10, in a case where the cyst 28 is recognized by executing the cyst recognition processing 91 and the blood vessel 29 is erroneously recognized as the cyst 28, the cyst recognition result 92 is superimposed on the ultrasound image 24 as the first bounding box BB1a and the second bounding box BB1b. The first bounding box BB1a is superimposed to surround a portion in the ultrasound image 24 where the cyst 28 is shown, and the second bounding box BB1b is superimposed to surround a portion in the ultrasound image 24 where the blood vessel 29 is shown.

However, in a case where the ultrasound image 24 in this state is displayed on the screen 26 as it is, there is a concern that the blood vessel 29 is erroneously recognized as the cyst 28 by the user 20.

Therefore, in the endoscope system 10, the Doppler image 88 associated with the ultrasound image 24 and the ultrasound image 24 in which the first bounding box BB1a and the second bounding box BB1b are superimposed are compared to each other to specify a high-intensity corresponding portion. The high-intensity corresponding portion refers to a portion corresponding to a high-intensity portion in the ultrasound image 24 in which the first bounding box BB1a and the second bounding box BB1b are superimposed. The high-intensity portion refers to a portion where the intensity of the blood flow is equal to or greater than the reference intensity TH in the Doppler image 88. In the endoscope system 10, in a case where the second bounding box BB1b is superimposed at the position surrounding the high-intensity corresponding portion, the second bounding box BB1b is erased. Then, the ultrasound image 24 on which the first bounding box BB1a is superimposed is displayed on the screen 26.

As a result, in a case where the blood vessel 29 is erroneously recognized as the cyst 28 by the cyst recognition processing 91, it is possible to display a correct recognition result and to suppress display of an erroneous recognition result. That is, the user 20 is allowed to visually recognize the first bounding box BB1a superimposed on the ultrasound image 24 without perceiving the second bounding box BB1b. This means that the user 20 can visually understand the portion in the ultrasound image 24 where the cyst 28 is shown and portions other than the cyst 28, through the first bounding box BB1a. In addition, this also means that, in a case where the cyst 28 and the blood vessel 29 are shown in the ultrasound image 24 in a state of being mixed, the user 20 can visually distinguish between the cyst 28 and the blood vessel 29.

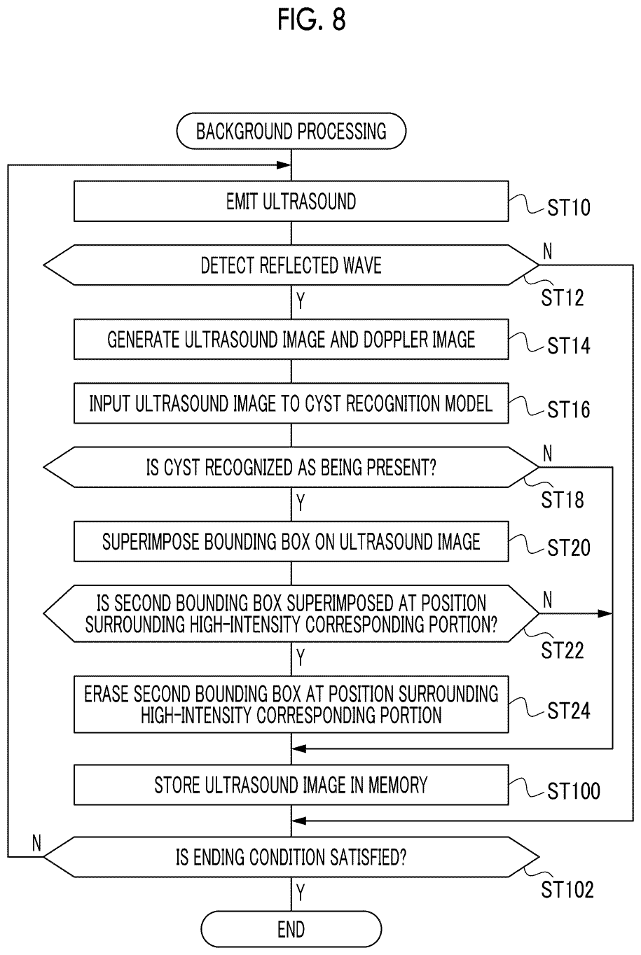

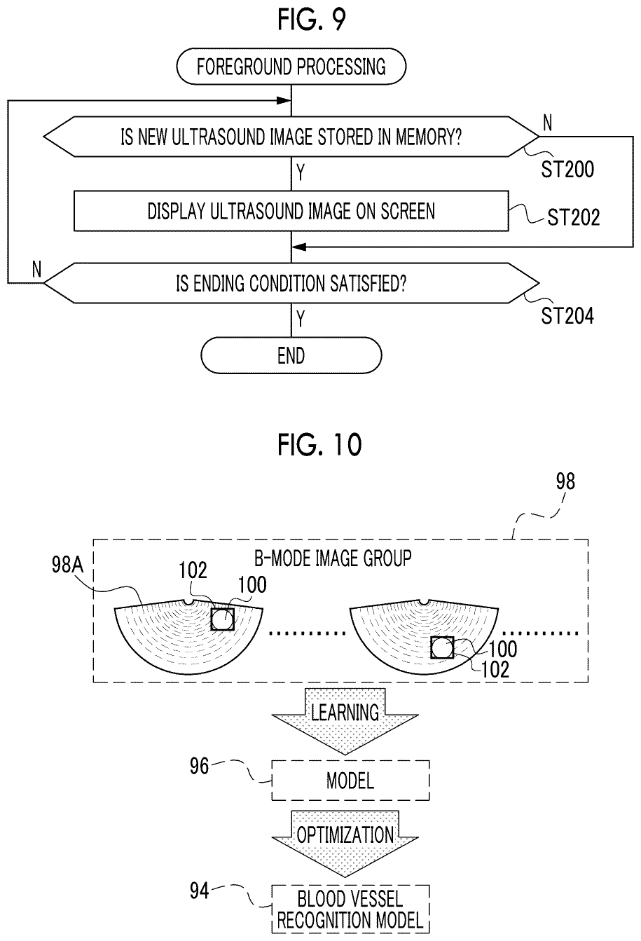

In the above-described embodiment, the form example has been described in which the diagnosis support processing is executed by sequential processing, but the diagnosis support processing may be implemented by parallel processing. For example, as shown in FIGS. 8 and 9, the same processing as the diagnosis support processing described in the above-described embodiment may be implemented by executing background processing and foreground processing in parallel. The background processing refers to processing in a so-called background, and the foreground processing refers to processing in a so-called foreground. For example, in the background processing, the cyst recognition processing 91 is executed, and the cyst recognition result 92 is changed based on the Doppler image 88, while in the foreground processing, the ultrasound image 24 is displayed on the screen 26.

Here, an example of a flow of the background processing will be described with reference to FIG. 8.

The background processing shown in FIG. 8 is different from the diagnosis support processing shown in FIG. 7 in that processing of step ST100 and processing of step ST102 are included instead of the processing of step ST26 and the processing of step ST28.

In step ST100 shown in FIG. 8, the control unit 62C stores the ultrasound image 24 in the memory 64. The ultrasound image 24 is stored in the memory 64 using a FIFO method. The memory 64 is also a buffer memory that can store a plurality of ultrasound images 24 in time series. In a case where a negative determination is made in step ST18, the processing of step ST100 is executed, and thus the ultrasound image 24 generated in step ST14 is stored in the memory 64. In addition, in a case where a negative determination is made in step ST22, the processing of step ST100 is executed, and thus the ultrasound image 24 on which the first bounding box BB1a is superimposed is stored in the memory 64. Furthermore, in a case where the diagnosis support processing proceeds to step ST100 after the processing of step ST24 is executed, the processing of step ST100 is executed, and thus the ultrasound image 24 on which the first bounding box BB1a is superimposed is stored in the memory 64. After the processing of step ST100 is executed, the diagnosis support processing proceeds to step ST102.

In step ST102, the control unit 62C determines whether or not a condition for ending the background processing is satisfied. Example of the condition for ending the background processing include a condition in which an instruction to end the background processing is received by the reception device 52. In step ST102, in a case where the condition for ending the background processing is not satisfied, a negative determination is made, and the background processing proceeds to step ST10. In step ST102, in a case where the condition for ending the background processing is satisfied, the background is ended.

Next, an example of a flow of the foreground processing will be described with reference to FIG. 9.

In step ST200 shown in FIG. 9, the control unit 62C determines whether or not a new ultrasound image 24 is stored in the memory 64. In step ST200, in a case where the new ultrasound image 24 is not stored in the memory 64, a negative determination is made, and the foreground processing proceeds to step ST202.

In step ST202, the control unit 62C acquires the ultrasound image 24 stored in the memory 64 (for example, the oldest ultrasound image 24 among the plurality of ultrasound images 24 stored in the memory 64 using the FIFO method) and displays the acquired ultrasound image 24 on the screen 26. After the processing of step ST202 is executed, the foreground processing proceeds to step ST204.

In step ST204, the control unit 62C determines whether or not a condition for ending the foreground processing is satisfied. Examples of the condition for ending the foreground processing include a condition in which an instruction to end the foreground processing is received by the reception device 52. In step ST204, in a case where the condition for ending the foreground processing is not satisfied, a negative determination is made, and the foreground processing proceeds to step ST200. In step ST204, in a case where the condition for ending the foreground processing is satisfied, the foreground is ended.

As described above, in the examples shown in FIGS. 8 and 9, in the background, the cyst recognition processing 91 is executed, and the cyst recognition result 92 is changed based on the Doppler image 88, while in the foreground, the ultrasound image 24 is displayed on the screen 26. In this way, the user 20 can visually recognize the ultrasound image 24 without perceiving that the cyst recognition processing 91 is executed or the cyst recognition result 92 is changed based on the Doppler image 88.

In the above-described embodiment, the form example has been described in which the cyst 28 is recognized and erroneous recognition of the blood vessel 29 as the cyst 28 is suppressed, but this is merely an example. For example, as shown in FIGS. 10 to 12, the blood vessel 29 may be recognized, and erroneous recognition of the cyst 28 as the blood vessel 29 may be suppressed. In order to implement this form example, a blood vessel recognition model 94 shown in FIG. 10 is used instead of the cyst recognition model 78. In the example shown in FIG. 10, the blood vessel recognition model 94 is an example of the “trained model” according to the present disclosure.

The blood vessel recognition model 94 is obtained by performing machine learning for recognizing the blood vessel 29 (see FIG. 1) shown in the ultrasound image 24 (see FIG. 1). As shown in FIG. 10 as an example, the blood vessel recognition model 94 is generated by causing a model 96 to perform machine learning. The model 96 has the same data structure as the model 80.

In the machine learning performed on the model 96, a B-mode image group 98 is used as training data. The B-mode image group 98 consists of a plurality of B-mode images 98A different from each other.

A blood vessel 100 is shown in the plurality of B-mode images 98A. An annotation 102 is assigned to the B-mode image 98A. The annotation 102 is information capable of specifying a position where the blood vessel 100 is shown in the B-mode image 98A (for example, information including a plurality of coordinates capable of specifying a position of a rectangular frame circumscribing the blood vessel 100).

Here, for convenience of description, as an example of the annotation 102, information capable of specifying the position where the blood vessel 100 is shown in the B-mode image 98A is shown, but this is merely an example. For example, the annotation 102 may include various types of information related to the blood vessel 100 shown in the B-mode image 98A, such as information capable of specifying a type of the blood vessel 100 shown in the B-mode image 98A, an importance of the blood vessel 100, and/or a size of the blood vessel 100.

It should be noted that, hereinafter, for convenience of description, processing using the blood vessel recognition model 94 will be described as processing that is actively performed by the blood vessel recognition model 94 as a main subject. That is, for convenience of description, the blood vessel recognition model 94 will be described as having a function of performing processing on input information and outputting a processing result. In addition, hereinafter, for convenience of description, a part of processing of training the model 96 will also be described as processing that is actively performed by the model 96 as a main subject. That is, for convenience of description, the model 96 will be described as having a function of performing processing on input information and outputting a processing result.

Each B-mode image 98A included in the B-mode image group 98 is input to the model 96. In response to this, the model 96 recognizes a position where the blood vessel 100 is shown from the input B-mode image 98A (in other words, predicts a position where the blood vessel 100 is shown). Then, the model 96 outputs a recognition result (in other words, a prediction result). The recognition result includes information capable of specifying the position recognized by the model 96 as the position where the blood vessel 100 is shown in the B-mode image 98A. Examples of the information capable of specifying the position recognized by the model 96 include information including a plurality of coordinates capable of specifying a position of a bounding box surrounding a region predicted as a position at which the blood vessel 100 is present (that is, the position of the bounding box in the B-mode image 98A).

The model 96 is adjusted according to an error between the annotation 102 assigned to the B-mode image 98A input to the model 96 and the recognition result output from the model 96. That is, the model 96 is optimized by adjusting a plurality of optimization variables (for example, a plurality of connection weights and a plurality of offset values) in the model 96 such that the error is minimized, whereby the blood vessel recognition model 94 is generated. That is, a data structure of the blood vessel recognition model 94 is obtained by causing the model 96 to learn the plurality of different B-mode images 98A to which the annotation 102 are assigned.

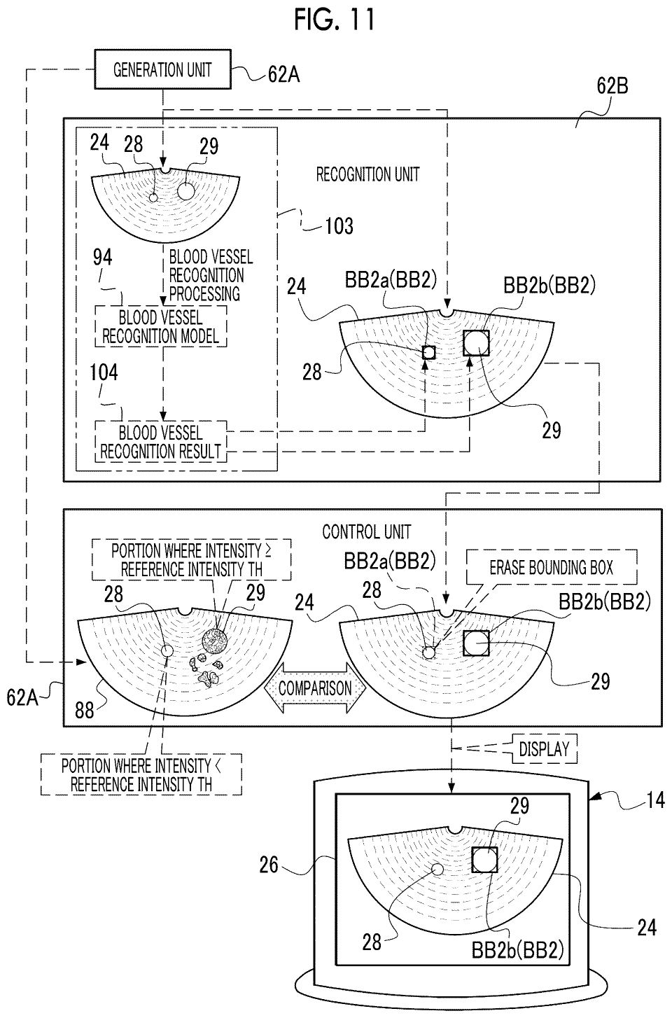

As shown in FIG. 11 as an example, the recognition unit 62B acquires the ultrasound image 24 generated by the generation unit 62A from the generation unit 62A, and executes blood vessel recognition processing 103 on the acquired ultrasound image 24. The blood vessel recognition processing 103 is processing of causing the blood vessel recognition model 94 to recognize the blood vessel 29 by inputting the ultrasound image 24 acquired from the generation unit 62A to the blood vessel recognition model 94. In the example shown in FIG. 11, the blood vessel recognition processing 103 is an example of the “recognition processing” according to the present disclosure. In addition, in the example shown in FIG. 11, the blood vessel 29 is an example of an “observation target region” and the “blood vessel” according to the present disclosure.

A blood vessel recognition result 104 is generated by the blood vessel recognition model 94 by executing the blood vessel recognition processing 103. The blood vessel recognition result 104 includes information capable of specifying the position of the blood vessel 29 shown in the ultrasound image 24. Here, the blood vessel recognition result 104 may include a result of erroneously recognizing the cyst 28 as the blood vessel 29.

The recognition unit 62B acquires the blood vessel recognition result 104 generated by the blood vessel recognition model 94. The recognition unit 62B reflects the blood vessel recognition result 104 on the ultrasound image 24 used in the blood vessel recognition processing 103. In this case, for example, the blood vessel recognition result 104 is reflected in the ultrasound image 24 as a bounding box BB2. A type of the bounding box BB2 is classified into a third bounding box BB2a and a fourth bounding box BB2b. The third bounding box BB2a is superimposed on a portion in the ultrasound image 24 where the cyst 28 is shown, by the recognition unit 62B. In the example shown in FIG. 11, the third bounding box BB2a is superimposed on the ultrasound image 24 as a circumscribing frame for the cyst 28 shown in the ultrasound image 24. The fourth bounding box BB2b is superimposed on a portion in the ultrasound image 24 where the blood vessel 29 is shown, by the recognition unit 62B. In the example shown in FIG. 11, the fourth bounding box BB2b is superimposed on the ultrasound image 24 as a circumscribing frame for the blood vessel 29 shown in the ultrasound image 24.

In addition, in the example shown in FIG. 11, a result of erroneously recognizing the cyst 28 as the blood vessel 29 is superimposed on the ultrasound image 24 as the third bounding box BB2a. Here, in a case where the ultrasound image 24 in which the third bounding box BB2a and the fourth bounding box BB2b are superimposed is displayed on the screen 26, there is a concern that the user 20 may erroneously recognize the cyst 28 as the blood vessel 29.

Therefore, in the present embodiment, the control unit 62C erases the third bounding box BB2a on the ultrasound image 24. In order to implement this, first, the control unit 62C acquires the ultrasound image 24 in which the third bounding box BB2a and the fourth bounding box BB2b are superimposed, from the recognition unit 62B. In addition, the control unit 62C acquires the Doppler image 88 (that is, the Doppler image 88 associated with the ultrasound image 24) generated in parallel with the ultrasound image 24 used in the blood vessel recognition processing 103 from the generation unit 62A, and compares the acquired Doppler image 88 to the ultrasound image 24 in which the third bounding box BB2a and the fourth bounding box BB2b are superimposed. Then, the control unit 62C specifies a portion (hereinafter, referred to as a “low-intensity portion”) in which the intensity of the blood flow is less than the reference intensity TH in the Doppler image 88.

The control unit 62C specifies a portion (hereinafter, referred to as a “low-intensity corresponding location”) corresponding to the low-intensity location in the ultrasound image 24 in which the third bounding box BB2a and the fourth bounding box BB2b are superimposed. In a case where the third bounding box BB2a is superimposed at a position surrounding the specified low-intensity corresponding portion, the control unit 62C erases the third bounding box BB2a. In other words, in the Doppler image 88, in a case where the intensity of the blood flow at the portion corresponding to the portion where the third bounding box BB2a is superimposed is less than the reference intensity TH, the control unit 62C erases the third bounding box BB2a. As a result, the ultrasound image 24 is in a state in which only the third bounding box BB2b of the third bounding box BB2a and the fourth bounding box BB2b is superimposed. This means that the blood vessel recognition result 104 reflected in the ultrasound image 24 is changed based on the Doppler image 88. That is, it can be said that the fourth bounding box BB2b remaining on the ultrasound image 24 by erasing the third bounding box BB2a from the ultrasound image 24 is information in which the blood vessel recognition result 104 is changed based on the low-intensity portion.

The fact that the third bounding box BB2a is erased from the ultrasound image 24 and the fourth bounding box BB2b remains means that the blood vessel 29 and portions other than the blood vessel 29 are distinguishable in the ultrasound image 24. In addition, the fact that the third bounding box BB2a is erased from the ultrasound image 24 and the fourth bounding box BB2b remains means that the blood vessel 29 is shown in a portion surrounded by the fourth bounding box BB2b in the ultrasound image 24, and there is a possibility that the cyst 28 is shown in a portion other than the portion surrounded by the fourth bounding box BB2b in the ultrasound image 24. This means that the blood vessel recognition result 104 is changed to distinguish between the cyst 28 and the blood vessel 29 based on the Doppler image 88. In other words, it can be said that the fourth bounding box BB2b remaining on the ultrasound image 24 by erasing the third bounding box BB2a from the ultrasound image 24 is information obtained by changing the blood vessel recognition result 104 to distinguish between the cyst 28 and the blood vessel 29 based on the Doppler image 88.

The control unit 62C outputs the ultrasound image 24 obtained by erasing the third bounding box BB2a from the ultrasound image 24 in which the third bounding box BB2a and the fourth bounding box BB2b are superimposed. In the example shown in FIG. 11, the ultrasound image 24 obtained by erasing the third bounding box BB2a from the ultrasound image 24 in which the third bounding box BB2a and the fourth bounding box BB2b are superimposed is displayed on the screen 26 by the control unit 62C. That is, the fourth bounding box BB2b superimposed on the ultrasound image 24 is displayed on the screen 26 as visible information for distinguishing between the blood vessel 29 and portions other than the blood vessel 29. Examples of the “portions other than the blood vessel 29” mentioned here include a region including the cyst 28 among regions shown in the ultrasound image 24.

Here, as an example of outputting the ultrasound image 24 on which the fourth bounding box BB2b is superimposed, an example in which the ultrasound image 24 on which the fourth bounding box BB2a is superimposed is displayed on the screen 26 has been described, but the present disclosure is not limited to this. For example, the ultrasound image 24 on which the fourth bounding box BB2b is superimposed may be stored in a storage region (the storage 66, a server, a personal computer, a tablet terminal, an electronic medical record, or the like), or may be recorded on a medium (for example, paper) by a printer (not shown). In addition, a position of the fourth bounding box BB2b (that is, the position of the blood vessel 29) in the ultrasound image 24 may be guided by a voice through a speaker (not shown).

In the present embodiment, the third bounding box BB2a and the fourth bounding box BB2b superimposed on the ultrasound image 24 are examples of the “first recognition result” according to the present disclosure. In addition, in the present embodiment, the fourth bounding box BB2b superimposed on the ultrasound image 24 displayed on the screen 26 is an example of the “second recognition result” and the “visible information” according to the present disclosure.

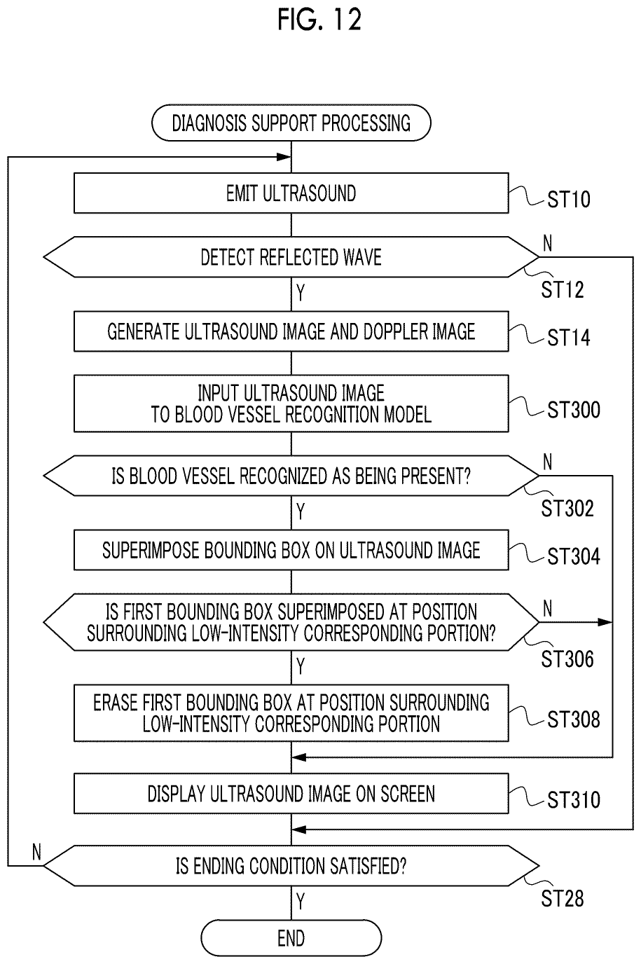

FIG. 12 shows a modification example of the flow of the diagnosis support processing performed by the processor 62 of the processing device 18, that is, an example of a flow of processing of recognizing the blood vessel 29 and suppressing erroneous recognition of the cyst 28 as the blood vessel 29. The flow of the diagnosis support processing shown in FIG. 12 is an example of the “image processing method” according to the present disclosure.

The flowchart shown in FIG. 12 is different from the flowchart shown in FIG. 7 in that processing of steps ST300 to ST310 is provided instead of the processing of steps ST16 to ST26.

In step ST300 shown in FIG. 12, the recognition unit 62B inputs the ultrasound image 24 generated in step ST14 to the blood vessel recognition model 94. After the processing of step ST300 is executed, the diagnosis support processing proceeds to step ST302.

In step ST302, the recognition unit 62B determines whether or not the blood vessel 29 is recognized as being shown in the ultrasound image 24 by the blood vessel recognition model 94 based on the input of the ultrasound image 24 to the blood vessel recognition model 94. In step ST302, in a case where the blood vessel 29 is not recognized as being shown in the ultrasound image 24, a negative determination is made, and the diagnosis support processing proceeds to step ST306. In step ST302, in a case where the blood vessel 29 is recognized as being shown in the ultrasound image 24, a positive determination is made, and the diagnosis support processing proceeds to step ST304.

In step ST304, the recognition unit 62B superimposes the blood vessel recognition result 104 as the bounding box BB2 on the ultrasound image 24 generated in step ST14. After the processing of step ST304 is executed, the diagnosis support processing proceeds to step ST306.

In step ST306, the control unit 62C determines whether or not the third bounding box BB2a is superimposed at a position surrounding the low-intensity corresponding portion in the ultrasound image 24 by comparing the Doppler image 88 generated in step ST14 to the ultrasound image 24 on which the bounding box BB2 is superimposed. In step ST306, in a case where the third bounding box BB2a is not superimposed at the position surrounding the low-intensity corresponding portion in the ultrasound image 24, a negative determination is made, and the diagnosis support processing proceeds to step ST310. In step ST306, in a case where the third bounding box BB2a is superimposed at the position surrounding the low-intensity corresponding portion in the ultrasound image 24, a positive determination is made, and the diagnosis support processing proceeds to step ST308.

In step ST308, the control unit 62C erases the third bounding box BB2a from the ultrasound image 24 on which the bounding box BB2 is superimposed. After the processing of step ST304 is executed, the diagnosis support processing proceeds to step ST310.

In step ST310, the control unit 62C displays the ultrasound image 24 on the screen 26. That is, in a case where a negative determination is made in step ST302, the ultrasound image 24 generated in step ST14 is displayed on the screen 26 by executing the processing of step ST310. In addition, in a case where a negative determination is made in step ST306, the ultrasound image 24 on which the fourth bounding box BB2b is superimposed is displayed on the screen 26 by executing the processing of step ST310. Furthermore, in a case where the diagnosis support processing has proceeded to step ST310 after the processing of step ST308 is executed, the ultrasound image 24 on which the fourth bounding box BB2b is superimposed is displayed on the screen 26 by executing the processing of step ST310. After the processing of step ST310 is executed, the diagnosis support processing proceeds to step ST28.

As described above, in the examples shown in FIGS. 10 to 12, in a case where the blood vessel 29 is recognized by executing the blood vessel recognition processing 103 and the cyst 28 is erroneously recognized as the blood vessel 29, the blood vessel recognition result 104 is superimposed on the ultrasound image 24 as the third bounding box BB2a and the fourth bounding box BB2b. The third bounding box BB2a is superimposed to surround a portion in the ultrasound image 24 where the cyst 28 is shown, and the fourth bounding box BB2b is superimposed to surround a portion in the ultrasound image 24 where the blood vessel 29 is shown.

However, in a case where the ultrasound image 24 in this state is displayed on the screen 26 as it is, there is a concern that the cyst 28 is erroneously recognized as the blood vessel 29 by the user 20.

Therefore, in the endoscope system 10, the Doppler image 88 associated with the ultrasound image 24 and the ultrasound image 24 in which the third bounding box BB2a and the fourth bounding box BB2b are superimposed are compared to each other to specify a low-intensity corresponding portion. The low intensity corresponding portion refers to a portion corresponding to a low-intensity portion in the ultrasound image 24 in which the third bounding box BB2a and the fourth bounding box BB2b are superimposed. The low-intensity portion refers to a portion where the intensity of the blood flow is less than the reference intensity TH in the Doppler image 88. In the endoscope system 10, in a case where the third bounding box BB2a is superimposed at the position surrounding the low-intensity corresponding portion, the third bounding box BB2a is erased. Then, the ultrasound image 24 on which the fourth bounding box BB2b is superimposed is displayed on the screen 26.

As a result, in a case where the cyst 28 is erroneously recognized as the blood vessel 29 by the blood vessel recognition processing 103, it is possible to display a correct recognition result and to suppress display of an erroneous recognition result. That is, the user 20 is allowed to visually recognize the fourth bounding box BB2b superimposed on the ultrasound image 24 without perceiving the third bounding box BB2a. In other words, the user 20 can visually understand the portion in the ultrasound image 24 where the blood vessel 29 is shown through the fourth bounding box BB2b. This also means that this can contribute to enabling the user 20 to visually distinguish between the cyst 28 and the blood vessel 29 in a case where the cyst 28 and the blood vessel 29 are shown in the ultrasound image 24 in a state of being mixed.

Although the form example has been described in which the diagnosis support processing shown in FIG. 12 is executed instead of the diagnosis support processing shown in FIG. 7, this is merely an example. For example, the diagnosis support processing shown in FIG. 7 and the diagnosis support processing shown in FIG. 12 may be performed in parallel. In this case, the first bounding box BB1a and the fourth bounding box BB2b may be displayed on the screen 26 in a visually distinguishable manner (for example, color, brightness, thickness, and/or line type). In addition, the first bounding box BB1a and the fourth bounding box BB2b may be displayed in a display aspect in which meanings of the first bounding box BB1a and the fourth bounding box BB2b are each specifiable. For example, the first bounding box BB1a and the fourth bounding box BB2b may be displayed in a display aspect in which the first bounding box BB1a, which is a bounding box capable of specifying the position of the cyst 28, is distinguished from the fourth bounding box BB2b, which is a bounding box capable of specifying the position of the blood vessel 29. In addition, information (for example, an identifier) capable of visually specifying that the first bounding box BB1a is information capable of specifying the position of the cyst 28 and the fourth bounding box BB2b is information capable of specify the position of the blood vessel 29 may be displayed.

In the above-described embodiment, although the form example has been described in which the second bounding box BB1b is erased, this is merely an example. For example, the second bounding box BB1b may be displayed on the screen 26 at a display level that is visually imperceptible. In addition, the first bounding box BB1a and the second bounding box BB1b may be displayed on the screen 26 in a display aspect in which the first bounding box BB1a, which is information capable of specifying the position of the cyst 28, can be visually distinguishable from the second bounding box BB1b, which is information capable of specifying the position of the blood vessel 29. For example, color and/or brightness may be changed between the first bounding box BB1a and the second bounding box BB1b, an identifier that is capable of specifying that the first bounding box BB1a is the bounding box BB1 indicating the position of the cyst 28 may be assigned to the first bounding box BB1a, and an identifier that is capable of specifying that the second bounding box BB1b is the bounding box BB1 indicating the position of the blood vessel 29 may be assigned to the second bounding box BB1b. The same applies to a relationship between the third bounding box BB2a and the fourth bounding box BB2b shown in FIG. 11.

In the above-described embodiment, the Doppler image 88 is used as the image to be compared to the ultrasound image 24, but the present disclosure is not limited to this. The color map 90 may be used instead of the Doppler image 88. This is because the control unit 62C can specify the presence or absence of the blood flow and the intensity of the blood flow from the color map 90, and thus, as in the above-described embodiment, the control unit 62C can specify a portion where “intensity≥reference intensity TH” is established, that is, the high-intensity portion, or can specify a portion where “intensity<reference intensity TH” is established, that is, the low-intensity portion.

In the above-described embodiment, the trained model for object recognition using a bounding box method with AI has been described as an example, but the present disclosure is also established by using a trained model for object recognition using a segmentation method with AI instead of the trained model for object recognition using the bounding box method with AI or together with the trained model for object recognition using the bounding box method with AI.

In the above-described embodiment, the form example has been described in which the ultrasound image 24 and the Doppler image 88 are generated based on the reflected wave detected by the ultrasound probe 38 of the ultrasound endoscope 12, but this is merely an example, and even in a case where an ultrasound image and a Doppler image are generated based on a reflected wave detected by an extracorporeal ultrasound probe, the diagnosis support processing can be executed in the same manner as in the above-described embodiment.

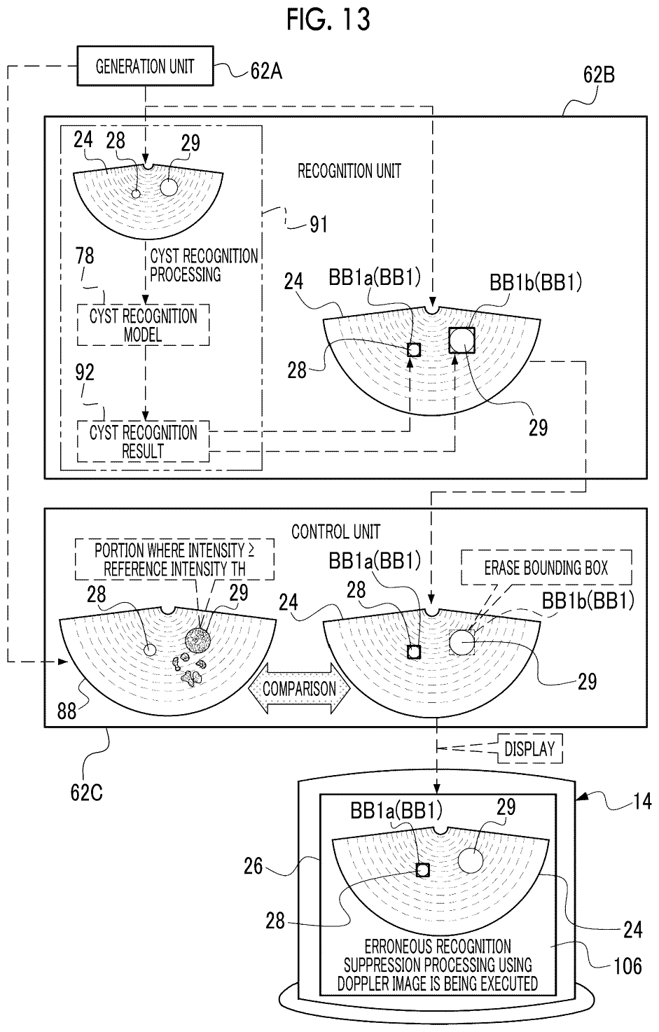

In the above-described embodiment, the form example has been described in which the ultrasound image 24 is displayed on the screen 26. However, for example, as shown in FIG. 13, in a case where the cyst recognition processing 91 is being executed, information 106 indicating that the processing of suppressing the erroneous recognition using the Doppler image 88 is being executed may be displayed on the screen 26, may be output as a voice, or may be stored in a storage region (the storage 66, a server, a personal computer, a tablet terminal, an electronic medical record, or the like). In the example shown in FIG. 13, as an example of the information 106, text indicating that the processing of suppressing erroneous recognition using the Doppler image 88 (in the example shown in FIG. 13, the cyst recognition processing 91) is being executed is displayed on the screen 26. As a result, the user 20 can recognize that the processing of suppressing erroneous recognition using the Doppler image 88 is being executed. In a case where the blood vessel recognition processing 103 is being executed, text indicating that the blood vessel recognition processing 103 is being executed may be displayed on the screen 26.

In addition, in the examples shown in FIGS. 8 and 9, in a case where the background processing is executed, in the foreground processing, information indicating that the background processing is being executed may be displayed on the screen 26, may be output as a voice, or may be stored in the storage region. The fact that the background processing is being executed refers to displaying the correct recognition result while suppressing display of the erroneous recognition result in the background (in other words, the cyst recognition processing 91 and/or the blood vessel recognition processing 103 is being executed in the background).

In the above-described embodiment, the generation of the ultrasound image 24 using the B-mode method and the generation of the Doppler image 88 using the Doppler method are performed in parallel, and the ultrasound image 24 and the Doppler image 88 generated at the same time are associated with each other. However, this is merely an example. For example, the generation of the ultrasound image 24 using the B-mode method and the generation of the Doppler image 88 using the Doppler method may be alternately performed, and the ultrasound image 24 and the Doppler image 88 generated in successive frames may be associated with each other.

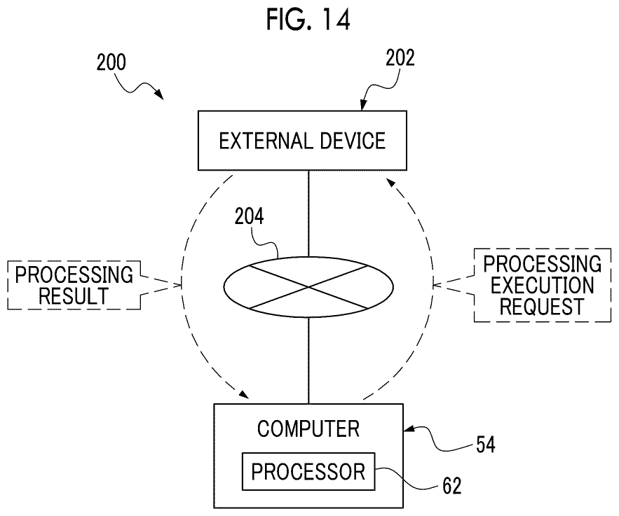

In the embodiment described above, the form example has been described in which the diagnosis support processing is performed by the computer 54, but the present disclosure is not limited to this. At least a part of processing included in the diagnosis support processing may be performed by a device provided outside the computer 54. Hereinafter, an example of this case will be described with reference to FIG. 14.

FIG. 14 is a conceptual diagram showing an example of a configuration of the endoscope system 200. The endoscope system 200 is an example of an “endoscope system” according to the present disclosure. The endoscope system 200 is different from the endoscope system 10 described in the above-described embodiment in that an external device 202 is provided.

The external device 202 is communicatively connected to the computer 54 via a network 204 (for example, a WAN and/or a LAN).

Examples of the external device 202 include at least one server that performs data transmission and reception directly or indirectly with the computer 54 via the network 204. The external device 202 receives a processing execution instruction given from the processor 62 of the computer 54 via the network 204. Then, the external device 202 executes processing according to the received processing execution instruction and transmits a processing result to the computer 54 via the network 204. In the computer 54, the processor 62 receives the processing result transmitted from the external device 202 via the network 204 and executes processing using the received processing result.

Examples of the processing execution instruction include an instruction for the external device 202 to execute at least a part of the diagnosis support processing. A first example of at least a part of the diagnosis support processing (that is, processing executed on the external device 202) includes the cyst recognition processing 91 and/or the blood vessel recognition processing 103 described above. In this case, the external device 202 executes the cyst recognition processing 91 and/or the blood vessel recognition processing 103 in response to the processing execution instruction given from the processor 62 via the network 204, and transmits a recognition result (for example, the cyst recognition result 92 and/or the blood vessel recognition result 104) to the computer 54 via the network 204. In the computer 54, the processor 62 receives the recognition result and executes the same processing as in the above-described embodiment by using the received recognition result.

A second example of at least a part of the diagnosis support processing (that is, processing executed on the external device 202) includes a part of the processing performed by the control unit 62C. Examples of a part of the processing performed by the control unit 62C include processing of comparing the ultrasound image 24 to the Doppler image 88 and erasing an unnecessary bounding box based on a comparison result. In this case, the external device 202 executes the processing of comparing the ultrasound image 24 to the Doppler image 88 and erasing the unnecessary bounding box based on the comparison result in response to the processing execution instruction given from the processor 62 via the network 204, and transmits a processing result (for example, the ultrasound image 24 from which the unnecessary bounding box is erased) to the computer 54 via the network 204. In the computer 54, the processor 62 receives the processing result and executes the same processing as in the above-described embodiment using the received processing result.

For example, the external device 202 is implemented by cloud computing. It should be noted that the cloud computing is merely an example, and the external device 202 may be implemented by network computing such as fog computing, edge computing, or grid computing. Instead of the server, at least one personal computer or the like may be used as the external device 202. In addition, a computing device having a communication function equipped with a plurality of types of AI functions may be used.

In the above-described embodiment, the form example has been described in which the diagnosis support program 76 is stored in the storage 66, but the present disclosure is not limited to this. For example, the diagnosis support program 76 may be stored in a portable computer-readable non-transitory storage medium, such as an SSD or a USB memory. The diagnosis support program 76 stored in the non-transitory storage medium is installed in the computer 54 of the endoscope system 10. The processor 62 executes the diagnosis support processing according to the diagnosis support program 76.

In addition, the diagnosis support program 76 may be stored in a storage device of another computer, a server, or the like that is connected to the endoscope system 10 via the network. Then, the diagnosis support program 76 may be downloaded and installed in the computer 54 in response to a request from the endoscope system 10.

It is not necessary to store all of the diagnosis support program 76 in a storage device such as another computer or a server device connected to the endoscope system 10 or to store all of the diagnosis support program 76 in the storage 66, and only a part of the diagnosis support program 76 may be stored.

The following various processors can be used as a hardware resource for executing the diagnosis support processing. Examples of the processor include a CPU that is a general-purpose processor that executes software, that is, a program, to function as the hardware resource executing the diagnosis support process. Examples of the processor also include a dedicated electric circuit that is a processor having a dedicated circuit configuration designed to execute specific processing, such as an FPGA, a PLD, or an ASIC. A memory is built in or connected to any processor, and any processor executes the diagnosis support processing using the memory.

The hardware resource for executing the diagnosis support processing may be configured by one of the various processors or by a combination of two or more processors of the same type or different types (for example, a combination of a plurality of FPGAs or a combination of a CPU and an FPGA). The hardware resource for executing the diagnosis support processing may also be one processor.

A first example of the configuration in which the hardware resource is configured by one processor includes a form in which one processor is configured by a combination of one or more CPUs and software, and this processor functions as the hardware resource for executing the diagnosis support processing. As a second example, as typified by a SoC or the like, there is a form in which a processor that implements all functions of a system including a plurality of hardware resources executing the diagnosis support processing with one IC chip is used. As described above, the diagnosis support processing is implemented using one or more of the various processors as the hardware resource.

More specifically, an electric circuit in which circuit elements such as semiconductor elements are combined can be used as a hardware structure of the various processors. In addition, the above-described diagnosis support processing is merely an example. Therefore, it goes without saying that unnecessary steps may be deleted, new steps may be added, or the processing order may be changed within a range that does not deviate from the gist.

The above-described contents and the above-shown contents are the detailed description of the parts according to the present disclosure, and are merely examples of the present disclosure. For example, the description of the configuration, the function, the operation, and the effect are the description of examples of the configuration, the function, the action, and the effect of the parts according to the present disclosure. Accordingly, it goes without saying that unnecessary parts may be deleted, new elements may be added, or replacements may be made with respect to the above-described contents and the above-shown contents within a range that does not deviate from the gist of the present disclosure. In addition, in order to avoid complications and facilitate understanding of the parts according to the present disclosure, the description of common technical knowledge or the like, which does not particularly require the description for enabling the implementation of the present disclosure, is omitted in the above-described contents and the above-shown contents.

All of the documents, the patent applications, and the technical standards described in the present specification are incorporated into the present specification by reference to the same extent as in a case in which the individual documents, patent applications, and technical standards are specifically and individually stated to be described by reference.

What is claimed is:

1. An image processing device comprising:
a processor,
wherein the processor is configured to:
acquire a first recognition result obtained by executing recognition processing of inputting an ultrasound image, which is obtained by a B-mode method in an ultrasound diagnosis for a subject and in which an observation target region of the subject is shown, to a trained model to cause the trained model to recognize the observation target region, and
output a second recognition result in which the first recognition result is changed based on blood flow distribution information obtained by a Doppler method used in combination with the B-mode method in the ultrasound diagnosis.

2. The image processing device according to claim 1,
wherein the observation target region is a lesion, and
the second recognition result is information obtained by changing the first recognition result such that the lesion and a portion other than the lesion are distinguishable from each other.

3. The image processing device according to claim 2,
wherein the lesion is a cyst.

4. The image processing device according to claim 2,
wherein the blood flow distribution information is a map capable of specifying an intensity of a blood flow, and
the second recognition result is information in which the first recognition result is changed based on a portion in the map where the intensity is equal to or greater than a reference intensity.

5. The image processing device according to claim 2,
wherein the trained model is obtained by performing machine learning for recognizing the lesion.

6. The image processing device according to claim 2,
wherein the second recognition result is information capable of distinguishing the lesion from the portion other than the lesion.

7. The image processing device according to claim 2,
wherein the outputting of the second recognition result includes displaying the second recognition result on a first screen as visible information capable of distinguishing the lesion from the portion other than the lesion.

8. The image processing device according to claim 1,
wherein the observation target region is a blood vessel,
the blood vessel is specified from the blood flow distribution information, and
the second recognition result is information obtained by changing the first recognition result such that the blood vessel and a portion other than the blood vessel are distinguishable from each other.

9. The image processing device according to claim 8,
wherein a lesion other than the blood vessel is included, and
the lesion is a cyst.

10. The image processing device according to claim 8,
wherein the blood flow distribution information is a map capable of specifying an intensity of a blood flow, and
the second recognition result is information in which the first recognition result is changed based on a portion in the map where the intensity is less than a reference intensity.

11. The image processing device according to claim 8,
wherein the trained model is obtained by performing machine learning for recognizing the blood vessel.

12. The image processing device according to claim 8,
wherein the second recognition result is information capable of distinguishing the blood vessel from the portion other than the blood vessel.

13. The image processing device according to claim 8,
wherein the outputting of the second recognition result includes displaying the second recognition result on a first screen as visible information capable of distinguishing the blood vessel from the portion other than the blood vessel.

14. The image processing device according to claim 1,
wherein the ultrasound image is displayed on a second screen in a foreground, and
the recognition processing is executed and the first recognition result is changed to the second recognition result in a background.

15. The image processing device according to claim 1,
wherein, in a case where processing of changing the first recognition result to the second recognition result based on the blood flow distribution information is being executed, the processor is configured to output information capable of specifying that the processing of changing the first recognition result to the second recognition result based on the blood flow distribution information is being executed.

16. An endoscope system comprising:
the image processing device according to claim 1; and
an ultrasound probe that emits ultrasound and detects a reflected wave of the ultrasound in a state in which the ultrasound probe is inserted into a body of the subject,
wherein the ultrasound image and the blood flow distribution information are generated based on the reflected wave detected by the ultrasound probe.

17. An image processing method comprising:
acquiring a first recognition result obtained by executing recognition processing of inputting an ultrasound image, which is obtained by a B-mode method in an ultrasound diagnosis for a subject and in which an observation target region of the subject is shown, to a trained model to cause the trained model to recognize the observation target region; and
outputting a second recognition result in which the first recognition result is changed based on blood flow distribution information obtained by a Doppler method used in combination with the B-mode method in the ultrasound diagnosis.

18. A non-transitory computer-readable storage medium storing a program executable by a computer to execute a process comprising:
acquiring a first recognition result obtained by executing recognition processing of inputting an ultrasound image, which is obtained by a B-mode method in an ultrasound diagnosis for a subject and in which an observation target region of the subject is shown, to a trained model to cause the trained model to recognize the observation target region; and
outputting a second recognition result in which the first recognition result is changed based on blood flow distribution information obtained by a Doppler method used in combination with the B-mode method in the ultrasound diagnosis.
